(19) United States
(12) Patent Application Publication
(10) Pub. No.: US 2012/0088271 A1
(43) Pub. Date: Apr. 12, 2012

(54) Use of an Endogenous 2-Micron Yeast Plasmid for Gene Over Expression

(75) Inventors: Farzad Haerizadeh, San Diego, CA (US); Fernando Valle, Burlingame, CA (US); Guillaume Cottarel, Mountain View, CA (US)

(73) Assignee: Codexis, Inc., Redwood City, CA (US)

(21) Appl. No.: 13/249,219

(22) Filed: Sep. 29, 2011

Related U.S. Application Data
(60) Provisional application No. 61/404,409, filed on Sep. 30, 2010.

(51) Int. Cl. C12P 21/06; C12N 1/19; C12N 15/63; C12N 15/64

(52) U.S. Cl. 435/69.1; 435/91.2; 435/91.4; 435/254.2; 435/254.21; 435/320.1

(57) ABSTRACT

Methods and compositions for making stable recombinant yeast 2 μm plasmids are provided. Homologous recombination is performed to clone a nucleic acid of interest into the yeast 2 μm plasmid. Heterologous nucleic acid subsequences are recombined between an FLP and a REP2 gene of the plasmid.

### CROSS-REFERENCE TO RELATED APPLICATION

This application claims priority to and benefit of U.S. Provisional Patent Application Ser. No. 61/404,409, filed on Sep. 30, 2010, the contents of which are hereby incorporated by reference in their entirety for all purposes.

### FIELD OF THE INVENTION

This invention is in the field of yeast cloning and expression, particularly as it applies to directed evolution.

### BACKGROUND OF THE INVENTION

Large combinatorial libraries of molecule variants are constructed and screened to generate and identify molecules, e.g., polypeptides or RNAs, with new or improved activities. Directed evolution approaches to combinatorial library construction can include, e.g., one or more rounds of random or directed combinatorial library construction, expression of library expression products in a suitable host, and screening of libraries of variant molecules for a property of interest. For a review of directed evolution and other combinatorial mutational approaches see, e.g., Brouk et al. (2010) “Improving Biocatalyst Performance by Integrating Statistical Methods into Protein Engineering,” Appl Environ Microbiol doi:10.1128/AEM.00878-10; Turner (2009) “Directed evolution drives the next generation of biocatalysts” Nat Chem Biol 5: 567-573; Fox and Huisman (2008), “Enzyme optimization: moving from blind evolution to statistical exploration of sequence-function space,” Trends Biotechnol 26: 132-138; Reetz et al. (2008) “Addressing the Numbers Problem in Directed Evolution,” ChemBioChem 9: 1797-1804; Arndt and Miller (2007) Methods in Molecular Biology, Vol. 352: Protein Engineering Protocols, Humana; Zhao (2006) Comb Chem High Throughput Screening 9: 247-257; Bershtein et al. (2006) Nature 444: 929-932; Brakmann and Schwienhorst (2004) Evolutionary Methods in Biotechnology: Clever Tricks for Directed Evolution, Wiley-VCH, Weinheim; Arnold and Georgiou (2003) Directed Evolution Library Creation Methods in Molecular Biology 231 Humana, Totowa; and Rubin-Pitel Arnold and Georgiou (2003) Directed Enzyme Evolution: Screening and Selection Methods, 230, Humana, Totowa.

One difficulty encountered in making combinatorial libraries is the high-throughput cloning and expression of molecular variants, particularly in eukaryotic cells. Typically, many eukaryotic expression libraries are initially cloned in prokaryotic cells, such as E. coli, as the methods for, e.g., nucleic acid manipulation and protein expression, in bacteria are both technically straightforward and well known in the art. However, many proteins and other expression products are not correctly processed (e.g., properly folded, inserted into the cell membrane or a subcellular structure, glycosylated, phosphorylated, prenylated, farnesylated, or the like) in prokaryotes or are otherwise not active in prokaryotic cells or cell extracts. As a result, many expression libraries are initially cloned in prokaryotic cells, such as E. coli, where cloning procedures are relatively straightforward, and then “shuttled” into a eukaryotic cell of interest, such as a yeast, fungal, mammalian, or insect cell for expression and screening.

Yeast and fungi represent one relatively well-established system for gene expression, e.g., subsequent to gene shuttling of clones from bacterial cells, using vectors that replicate in both prokaryotes and eukaryotes. For example, yeast can be transformed by various shuttle plasmids that are replication competent in both yeast and E. coli. For an introduction to the topic of shuttle vectors and expression of proteins in yeast and other eukaryotes, see, e.g., Amberg et al. (2005) Methods in Yeast Genetics: A Cold Spring Harbor Laboratory Course Manual Cold Spring Harbor Laboratory Press ISBN-10: 0879697288 (ISBN-13: 978-0879697280); Baneyx (ed) (2004) Protein Expression Technologies: Current Status and Future Trends (Horizon Bioscience) ISBN-10: 0954523253 (ISBN-13: 978-0954523251); and Demian et al. (1999) Manual of Industrial Microbiology and Biotechnology ISBN-10: 1555811280 (ISBN-13: 978-1555811280) and Romanos et al. (1992) “Foreign Gene Expression in Yeast: a Review” YEAST 8: 423-488 (1992).

In one example, the endogenous yeast 2 μm plasmid of Saccharomyces cerevisiae has been used as the basis for various shuttle vectors. Such shuttle vectors include bacterial replication elements (for initial cloning and replication in bacterial cells), restriction enzyme cloning sites, and portions of the endogenous yeast 2 μm plasmid sufficient for replication in yeast. See, e.g., Amberg et al. (2005) above; Romanos et al. (1992) above; Soni et al. (1992) “A rapid and inexpensive method for isolation of shuttle vector DNA from yeast for the transformation of E. coli.” Nucl Acids Res 20: 5852; and Armstrong et al. (1989) “Propagation and expression of genes in yeast using 2 μm circle vectors. In Barr, P. J., Brake, A. J. and Valenzuela, P. (Eds), Yeast Genetic Engineering. Butterworths, pp. 165-192. Various shuttle vectors are also proposed, e.g., in Hinchliffe et al. (1994) YEAST VECTOR EP 0286424B1; Hinchliffe et al. (1997) STABLE YEAST 2 μM VECTOR U.S. Pat. No. 5,637,504; and Sleep et al. 2 μM FAMILY PLASMID AND USE THEREOF US Patent Application Publication No. 2008/0261861. A difficulty in such prior art approaches, particularly as applied to combinatorial library generation, is the need to initially clone a gene of interest in bacteria, prior to transfer. In addition to the complexity of cloning and selecting genes in two different cell types (difficulties which can be compounded during the creation of complex combinatorial libraries), this approach suffers from the need for the shuttle vector to comprise a variety of elements to support cloning, replication in two separate cell types, etc. The different size and sequence constraints imposed by differing host cells can hamper cloning and vector stability. In addition, prior art approaches typically rely on the use of FLP recombination sites to remove any unwanted bacterial sequences once the vectors are shuttled into yeast, e.g., by adding copies of FLP sites flanking the bacterial sequences and relying on FLP-mediated recombination to remove bacterial sequences from the shuttle vector once the vector is propagated in yeast. This necessitates additional structural constraints on the shuttle vectors and on nucleic acids cloned into them for expression.

Another difficulty in screening expression libraries is that relatively low levels of a product of interest may be produced after shuttling into yeast. This has been addressed, e.g., by using yeast species that grow to very high culture densities, such as the methylotrophic yeast Pichia Pastoris. See, e.g., Lin-Cereghino, et al. (2000) “Heterologous protein expression in the methylotrophic yeast Pichia pastoris.” FEMS Microbiol Rev 24: 45-66; and Higgins and Cregg, (1999) Pichia Protocols (Methods in Molecular Biology Humana Press; 1st edition ISBN-10: 0896034216, ISBN-13: 978-0896034211. However, plasmid vectors are, in general, unstable in Pichia, necessitating the use of genomic recombination to incorporate a nucleic acid of interest. This has a variety of practical disadvantages, including limiting the copy number of a gene that can easily be incorporated into Pichia, and increased the complexity involved in transferring an incorporated gene out of Pichia.

New vectors and methods that facilitate high throughput cloning of nucleic acids of interest, e.g., in standard yeast systems such as Saccharomyces cerevisiae, would be desirable, e.g., in the context of combinatorial library production. Desirably, such systems would be capable of producing high levels of, e.g., a polypeptide or RNA of interest. The present invention provides these and other features.

### SUMMARY OF THE INVENTION

The invention provides methods and compositions for direct cloning of a molecule of interest into a mitotically stable extrachromosomal genetic element in a yeast cell or other fungal cell. In the methods, homologous recombination is performed to incorporate a nucleic acid of interest into endogenous or introduced nuclear or other plasmids such as the 2 μm plasmids, e.g., in yeast such as Saccharomyces, e.g., Saccharomyces cerevisiae, such as the strain NRLL YB-1952 (RN4). The invention also includes the surprising discovery of a site for homologous recombination between the FLP and REP2 genes of the 2 μm plasmid. Such direct cloning into a yeast plasmid, or other fungal plasmid, is advantageous because it eliminates any need for shuttling procedures between bacterial and eukaryotic cells, thereby permitting the facile construction of combinatorial libraries of molecule variants in fungi or yeast. This is particularly useful, e.g., where properties of interest of members of a combinatorial library can also be screened in the yeast or other fungi.

Accordingly, the invention provides compositions that include a stable recombinant yeast 2 μm or other nuclear or other endogenous plasmid that includes an introduced heterologous nucleic acid subsequence, e.g., between an FLP and a REP2 gene of the plasmid. The 2 μm or other plasmid can be, e.g., endogenous to the cell, or can be introduced into the cell. Example plasmids include those that have been sequenced, such as the endogenous plasmid for Saccharomyces cerevisiae strain RN4, e.g., SEQ ID NO: 1. Other suitable 2 μm plasmids include examples include Saccharomyces cerevisiae strain A364A (GeneBank J01347.1). For example, the plasmid can comprises a subsequence that is at least 90%, at least 91%, at least 92%, at least 93%, at least 94%, at least 95%, at least 96%, at least 97%, at least 98%, or at least 99% identical to a full-length endogenous 2 μm plasmid sequence from yeast RN4 or A364A (SEQ ID NO: 1; GeneBank J01347.1).

Typically, the plasmid is free of a bacterial origin of replication, because the methods of the invention do not rely on cloning in bacterial cells, or replication of vectors in bacteria. 2 μm plasmids optionally includes a complete set of native 2 μm plasmid coding and regulatory sequences, e.g., including sequences that encode functional REP1, REP2 and FLP proteins.

The heterologous nucleic acid typically encodes a selectable marker to facilitate selection during cloning, e.g., a hygromycin selectable marker or a nourseothricin selectable marker. The heterologous nucleic acid optionally additionally encodes a polypeptide or RNA product of interest (e.g., a coding sequence for an enzyme or other polypeptide, or a ribozyme, RNAi, or the like). The encoded polypeptide can optionally comprise an enzyme, e.g., a dehydrogenase, a dehydratase, or an invertase. Properties of the product of interest can also be selected, e.g., as part of the overall process of selecting members of a combinatorial library for a property of interest. For example, in one embodiment, the polypeptide or other product catalyzes or regulates degradation or synthesis of a sugar, a polysaccharide, a cellulosic material, a polymer, a chemical compound, a fatty acid, a fatty alcohol, a ketone, a lipid, an organic acid, or succinate. In another example, the polypeptide or target RNA product regulates expression, synthesis, or folding of an additional polypeptide that catalyzes or regulates degradation or synthesis of such an enzyme. The regulation, catalysis, degradation or other activity of the polypeptide, additional polypeptide or other product can be measured and selected for. Optionally, both the selectable marker and the product of interest can be selected for, e.g., in the yeast or fungal cell into which the heterologous nucleic acid is cloned. Markers and products can also be measured and selected for outside of the cells, e.g., in a cell extract or lysate, or, optionally, following subcloning and expression in an additional cell type.

Typically, the plasmid is stably propagated in a yeast cell culture comprising a selection agent, e.g., hygromycin, nourseothricin, etc., that selects for an expression product of the heterologous nucleic acid subsequence. Thus, compositions can include a yeast cell culture, e.g., optionally also including the selection agent and/or an expression product that has selection agent resistance activity. Typically, the selection agent is present in the composition at a concentration sufficient to exert selective pressure on cells of the culture, which assists in stably retaining the plasmid. Typical selection agents include antifungal agents, antibiotic agents, toxins, etc. Alternately, but equally preferred, the yeast cell culture can be an auxotrophic cell culture, with the plasmid encoding an auxotrophic agent that increases a rate of growth of cells in the culture under non-permissive auxotrophic growth conditions.

The invention includes yeast cells that include the plasmids described above and elsewhere herein. In typical embodiments, the cell can include at least about 5 copies of the plasmid, more preferably at least about 10 copies of the plasmid. Optionally, more than 10 copies are present per cell, e.g., about 20, about 30, about 40, about 50, about 60, about 70, about 80, about 90, or about 100 or more copies. The cell will typically be any fungal or yeast cell that supports replication of the yeast 2 μm plasmid, e.g., a Saccharomyces cell, such as, e.g., a Saccharomyces cerevisiae cell, such as a NRLL YB-1952 (RN4) cell.

The invention also includes methods of making a recombinant plasmid in a yeast or fungal cell. The method includes providing a yeast or fungal cell, e.g., a NRLL YB-1952 (RN4) cell, that includes a stable 2 μm plasmid and introducing a heterologous nucleic acid into the cell. The heterologous nucleic acid has recombination sites flanking a subsequence encoding a selectable marker. Integration of the selectable marker into the 2 μm plasmid is permitted via homologous recombination between the recombination sites and the plasmid, producing a recombinant plasmid in the cell. The 2 μm plasmid can be a wild-type 2 μm plasmid endogenous to the cell (e.g., an endogenous 2 μm plasmid of a Saccharomyces, e.g., a Saccharomyces cerevisiae cell, such as a NRLL YB-1952 (RN4) cell), or the method can include introducing the 2 μm plasmid into the yeast cell.

The method typically includes assembling the heterologous nucleic acid via PCR, by direct synthesis, or both. The heterologous nucleic acid can be produced, e.g., via PCR, LCR, splicing by overlap extenstion (SOE) PCR, direct synthesis, or other synthesis methods. These methods can be used alone or in combination. Homologous recombination occurs between subsequences of the 2 μm plasmid and the heterologous nucleic acid, e.g., at a site between the genes for FLP and REP2. The yeast cell can be propagated under selective conditions after integration, thereby selecting progeny of the yeast cell based upon expression of the selectable marker. Selective conditions can, optionally, be continuously maintained to facilitate selection and to increase stability of the plasmid during a growth phase of the yeast culture. Selective conditions can also act to raise copy number, by applying selective pressure for increased expression of a selectable marker.

In one embodiment, assembling the heterologous nucleic acid comprises amplifying a hygromycin resistance marker using primers encoded by SEQ ID NOs: 26 and 27. In an alternate embodiment, assembling the heterologous nucleic acid comprises amplifying a nourseothricin resistance marker, e.g., a Gene 1/Gateway/Sat 1 marker cassette, using primers encoded by SEQ ID NOs: 32 and 33.

Selective conditions optionally comprise non-permissive auxotrophic growth conditions, e.g., where the selectable marker includes an auxotrophic growth agent. Alternately, selective conditions can include culturing yeast cells harboring plasmids with the nucleic acid of interest in the presence of an antibiotic, an antifungal, or a toxin, e.g., where the selectable marker includes a resistance agent to the antibiotic, the antifungal, or the toxin. For example, in one convenient embodiment, the selectable marker provides hygromycin resistance to the yeast cell. In a second embodiment, the selectable marker provides nourseothricin resistance to the cell. In an alternate embodiment, counter selection markers can be used. These markers prevent growth in cells harboring an appropriate marker. An additional type of useful selection relies on selection of an introduced trait. For example, if the introduced nucleic acid encodes a visible marker, such as a red or green florescent protein, then cells can be selected by visual inspection. In yet an additional alternate embodiment, a marker can comprise a gene that encodes an agent that yields a selective advantage to the cell expressing the agent, e.g., the ability to more efficiently use an energy source in the culture medium.

Accordingly, the nucleic acid of interest comprises a selectable marker, e.g., a hygromycin selectable marker or a nourseothricin selectable marker. Culturing the yeast cell under selective conditions results in progeny yeast cells comprising at least about 5 copies, or at least about 10 copies of the recombinant plasmid (e.g., the yeast 2 μm plasmid comprising the nucleic acid of interest) per cell. Preferably, selection results in about 20, about 30, about 40, about 50, about 60, about 70, about 80, about 90, or about 100 or more copies per cell. Typical copy numbers can be, e.g., in the range of about 40 to about 60 copies per cell. In certain embodiments, culturing the yeast under selective conditions includes plating the yeast on YPD agar plates comprising 300 μg/ml hygromycin or YPD agar plates comprising 100 μg/ml nourseothricin

In some embodiments, the methods optionally include isolating copies of the recombinant plasmid from the progeny and introducing one or more of the copies into one or more additional cell(s). This procedure can be used to introduce the recombinant plasmid from a convenient cloning strain of yeast or fungi, into a cell that comprises traits that are useful for a particular application.

Typically, the heterologous nucleic acid includes a gene or expression cassette that encodes a polypeptide or RNA product of interest in addition to encoding the selectable marker. Optionally, the encoded polypeptide comprises an enzyme, e.g., a dehydrogenase, a dehydratase, or an invertase. In one aspect, the polypeptide or RNA product of interest optionally catalyzes or regulates degradation or synthesis of a sugar, a polysaccharide, a cellulosic material, a polymer, a chemical compound, a fatty acid, a fatty alcohol, a ketone, a lipid, an organic acid, or succinate.

Optionally, in one useful class of embodiments, the method includes introducing a pooled population of variant heterologous nucleic acids into a population of yeast cells, and selecting the population of yeast cells for one or more activity of interest. The pooled population of variant heterologous nucleic acids can be produced by any available combinatorial method, e.g., shuffling, LCR, PCR, SOE PCR, direct synthesis, or a combination thereof.

The invention also provides a method of producing a protein that comprises culturing a yeast cell made by the methods described above.

Kits and apparatus comprising the compositions are also a feature of the invention. Kits will typically include the compositions of the invention packaged for use. Such kits can include instructions regarding practicing the methods herein, e.g., using the compositions of the kit, and can additionally include standardization materials, e.g., control nucleic acids for integration, 2 μm plasmids, yeast cells, etc.

Those of skill in the art will appreciate that the methods and compositions provided by the invention can be used alone or in combination. Apparatus and systems are a feature of the invention can include any of the compositions or kits described above. Such apparatus and systems and can additionally include modules that perform the methods in an automated fashion, e.g., computer controllers linked to fluid handling elements that move or assemble the compositions of the invention.

These and other features of the invention will become more fully apparent when the following detailed description is read in conjunction with the accompanying figures and claims.

### DEFINITIONS

It is to be understood that this invention is not limited to particular systems, devices or biological systems, which can, of course, vary. It is also to be understood that the terminology used herein is for the purpose of describing particular embodiments only, and is not intended to be limiting. As used in this specification and the appended claims, the singular forms “a”, “an” and “the” optionally include plural referents unless the content clearly dictates otherwise. Thus, for example, reference to “a yeast cell” includes a combination of two or more cells (e.g., in a culture); reference to “bacteria” includes mixtures of bacteria, and the like.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein

An “endogenous” polynucleotide, gene, promoter or polypeptide refers to any polynucleotide, gene, promoter or polypeptide that originates in a particular host cell. A polynucleotide, gene, promoter or polypeptide is not endogenous to a host cell if it has been removed from the host cell, subjected to laboratory manipulation, and then reintroduced into a host cell.

A “heterologous” polynucleotide, gene, promoter or polypeptide refers to any polynucleotide, gene, promoter or polypeptide that is introduced into a host cell that is not normally present in that cell, and includes any polynucleotide, gene, promoter or polypeptide that is removed from the host cell and then reintroduced into the host cell. In certain embodiments, heterologous proteins and heterologous nucleic acids remain “functional”, i.e., retain their activity or exhibit an enhanced activity in the host cell.

“Non-permissive auxotrophic growth conditions” are culture conditions under which growth of an auxotrophic cell is inhibited. For example, if a cell lacks the ability to synthesize a selected amino acid, then non-permissive auxotrophic growth conditions would include culture of the cell without the selected amino acid in the growth media.

As used herein, the terms “peptide”, “polypeptide”, and “protein” are used interchangeably herein to refer to a polymer of amino acid residues.

As used herein, the term “recombinant” refers to a polynucleotide or polypeptide that does not naturally occur in a host cell. In some embodiments, recombinant nucleic acid molecules contain two or more naturally-occurring sequences that are linked together in a way that does not occur naturally. A recombinant protein refers to a protein that is encoded and/or expressed by a recombinant nucleic acid. In some embodiments, “recombinant cells” express genes that are not found in identical form within the native (i.e., non-recombinant) form of the cell and/or express native genes that are otherwise abnormally over-expressed, under-expressed, and/or not expressed at all due to deliberate human intervention. Recombinant cells contain at least one recombinant polynucleotide or polypeptide. A nucleic acid construct, nucleic acid (e.g., a polynucleotide), polypeptide, or host cell is referred to herein as “recombinant” when it is non-naturally occurring, artificial or engineered. “Recombination”, “recombining”, and generating a “recombined” nucleic acid generally encompass the assembly of at least two nucleic acid fragments. In certain embodiments, recombinant proteins and recombinant nucleic acids remain functional, i.e., retain their activity or exhibit an enhanced activity in the host cell.

A “stable” recombinant yeast 2 μm plasmid is a yeast 2 μm plasmid that displays at least 40%, at least 50%, at least 60%, at least 70%, or greater than 70% retention in a yeast cell culture under conditions selected to maintain the plasmid in the yeast cell culture. For example, where the yeast is an auxotrophic strain, and the plasmid encodes a selectable auxotrophic component that remedies a deficiency of the auxotrophic strain, the conditions can be those under which expression of the selectable auxotrophic component is necessary for growth of yeast cells in the culture, such that, e.g., at least 40%, at least 50%, at least 60%, at least 70%, or greater than 70% of the cells in the culture comprise the plasmid, e.g., during growth phase of the culture. Similarly, where the plasmid encodes a drug resistance component (e.g., an antibiotic or antifungal agent, or an antitoxin), the plasmid is stably retained under culture conditions where expression of the drug resistance component is necessary for growth or survival of the cells in the culture. In preferred embodiments, the plasmid is stable when at least about 90%, 95%, 99% or more of the yeast cells in culture comprise the plasmid under conditions selected to maintain the plasmid in the yeast cell culture.

A “variant” is a polypeptide or nucleic acid that differs from, e.g., a wild type polypeptide or nucleic acid, or, e.g., the polypeptide or nucleic acid from which the variant is derived, by one or more amino acid or nucleotide substitutions, one or more amino acid or nucleotide insertions, or one or more amino acid or nucleotide deletions. Additionally or alternatively, a “variant” polypeptide or nucleic acid can comprise a subsequence of the polypeptide or nucleic acid from which the variant is derived.

### BRIEF DESCRIPTION OF THE FIGURES

FIG. 1 is a schematic illustration showing 3 preferred insertion sites upstream of the FLP coding region in the native 2 μm plasmid from Saccharomyces cerevisiae.

FIG. 2 is a schematic illustration of the yeast 2 μm plasmid from Saccharomyces cerevisiae strain RN4.

FIG. 3 is a graph showing percent retention of recombinant 2 μm plasmid constructs in strain RN4.

FIG. 4 is a graph showing percent retention of recombinant 2 μm plasmid constructs in strain RN4.

### DETAILED DESCRIPTION

The invention provides methods and compositions that permit the direct cloning of nucleic acids of interest into mitotically stable endogenous yeast plasmids, e.g., the Saccharomyces cerevisiae 2 μm plasmid, or, e.g., vectors derived from endogenous plasmids. Typically, cloning in yeast requires a shuttle vector, i.e., a vector that can propagate in two different host species, i.e., E. coli and yeast. The initial cloning and selection is performed in E. coli, and following plasmid purification and characterization, the recombinant vector is then “shuttled” into a yeast cell host. However, many shuttle vectors contain just a few unique cloning sites. In addition, many shuttle vectors show low levels of mitotic stability, as the bacterial sequences present in shuttle vectors can inhibit vector replication in yeast.

In the present invention, nucleic acids of interest can be introduced into the 2 μm plasmid, or a vector based on the 2 μm plasmid, in a host yeast cell, i.e., via homologous recombination. Accordingly, the invention simplifies the cloning and expression of, e.g., polypeptides and RNAs, particularly in yeast such as Saccharomyces, e.g., Saccharomyces cerevisiae, or, e.g., Torulaspora delbrueckii, Kluyveromyces drosophilarum, Glomerella musae, Collectotrichium musae, etc., by eliminating the need to first clone sequences of interest in a bacterial host cell. Thus, in addition to the other features of 2 μm plasmids, the plasmids of the invention are free of bacterial sequences, e.g., sequences that are required for the propagation a shuttle vector in a prokaryotic host. In contrast, previously described plasmids for introducing heterologous nucleic acid sequences in yeast (see, e.g., Hinchliffe et al. (1994) YEAST VECTOR EP 0286424B1 and Hinchliffe et al. (1997) STABLE YEAST 2 μM VECTOR U.S. Pat. No. 5,637,504) comprise one or more bacterial plasmid sequences. Furthermore, because plasmids such as the 2 μm plasmid are endogenous to yeast, the yeast cells do not have to be co-transfected with vector sequences. In addition, the stability and high copy number of, e.g., the 2 μm plasmid, can be beneficial in increasing the expression levels of, e.g., proteins or RNAs of interest, in yeast, e.g., in Saccharomyces, e.g., in Saccharomyces cerevisiae. For example, the level of a polypeptide or RNA of interest expressed from a heterologous nucleic acid present on a plasmid described herein can be, e.g., at least 10% greater, at least 20% greater, at least 30% greater, at least 40% greater, at least 50% greater, at least 60% greater, at least 70% greater, at least 80% greater, at least 90% greater, at least 100% greater, or more than 100% greater than the level of the polypeptide or RNA of interest expressed from a heterologous nucleic acid that has been integrated into a yeast's genome.

A variety of applications for the invention are described herein, including, e.g., simplifying combinatorial library construction. This, in turn, is useful for directed evolution and/or development of polypeptides and RNAs of interest. Example applications of interest include the rapid evolution of enzymes or other polypeptides that catalyze or regulate degradation or synthesis of sugars, polysaccharides, cellulosic materials, polymers, chemical compounds, fatty acids, fatty alcohols, ketones, lipids, organic acids, succinate, etc. Additionally or alternatively, RNAs (e.g., siRNAs, catalytic RNAs, or the like) and factors that regulate expression of polypeptides of interest can be similarly screened.

One aspect of the invention is the discovery and sequencing of a new endogenous 2 μm plasmid from yeast strain RN4. RN4 was isolated from the Agricultural Research Service Culture Collection (NRRL) yeast strain YB-1952. YB-1952 is publicly available from NRRL. The strain is further described in Fay and Benavides (2005) “Hypervariable noncoding sequences in Saccharomyces cerevisiae,” Genetics 170: 1575-1587 and Fay and Benavides (2005) “Evidence for domesticated and wild populations of Saccharomyces cerevisiae,” PLoS Genet. 1:66-71.

### The Yeast 2 μM Vector and Homologous Recombination

The 2 μm plasmid is a 6,318-base pair double-stranded plasmid that is endogenous in most strains of Saccharomyces cerevisiae. The 2 μm plasmid exhibits a high level of mitotic stability, which makes the 2 μm plasmid an attractive target for development as a useful yeast vector in the context of the present invention. As discussed herein, the inherently high stability of this plasmid, and/or other endogenous yeast plasmids, can also be improved through appropriate selection methods that select for progeny that carry the plasmid.

Examples of 2 μm plasmids are described herein and in the art and can be used in the methods herein. For example, a complete 2 μm plasmid for Saccharomyces cerevisiae is found in GenBank, e.g., at accession number J01347.1. Additional examples are described herein, e.g., SEQ ID NO: 1.

Other known endogenous plasmids from yeast can similarly be used for stable expression, e.g., by recombining a nucleic acid of interest with the native yeast plasmid as described herein. For example, the circular plasmid pTD1 of Torulaspora delbrueckii can be used as an expression vector in essentially the same manner as described herein for the 2 μm plasmid. Further details regarding pTD1 can be found, e.g., in Blaisonneau et al. (1997) “A Circular Plasmid from the Yeast Torulaspora delbrueckii,” Plasmid 38: 202-209. The sequence for pTD1 is found in GenBank at accession number Y11042.1. Similarly, the yeast Kluyveromyces drosophilarum can harbor the native plasmid pKD1, which can be used as a homologous recombination vector as described herein. For a description of PKD1, see, e.g., Chen et al. (1986) “Sequence organization of the circular plasmid pKD1 from the yeast Kluyveromyces drosophilarum,” Nucleic Acids Res. 14: 4471-4481. Linear plasmids, e.g., those of filamentous fungi, can also be targeted for direct recombination, e.g., pGML1 from Glomerella musae. See, e.g., Freeman et al. (1997) “Characterization of a linear DNA plasmid from the filamentous fungal plant pathogen Glomerella musae [Anamorph: Colletotrichum musae (Berk. & Curt.) Arx.],” Curr Genet. 32: 152-156. In general, a wide variety of plasmids from filamentous fungi are known and available for use according to the present invention. For a review of plasmids in filamentous fungi, see, e.g., Griffiths (1995) “Natural Plasmids of Filamentous Fungi” in Microbiological Reviews, 59: 673-685.

Endogenous yeast plasmids, such as the 2 μm plasmid, are well characterized in the art, and this knowledge informs selection of sites for recombination in such plasmids, as well as appropriate propagation conditions, etc. The 2 μm plasmid, for example, exists in yeast as a circular multicopy plasmid in the nucleus of the Saccharomyces cerevisiae cell. At its typical steady-state copy number (i.e., approximately 40-100 copies per cell), the 2 μm plasmid propagates itself without either conferring a clear advantage to its host or posing a significant burden on host cell fitness, at least under typical culture conditions. See, e.g., Jayaram et al. (2004) “The 2 μm plasmid of Saccharomyces cerevisiae,” In Plasmid Biology Funnell and Phillips (Eds.). ASM Press, Washington, D.C. 303-323; Velmurugan et al. (2004) “Selfishness in moderation: evolutionary success of the yeast plasmid,” Curr Top Dev Biol 56: 1-24; Velmurugan et al. (2000) “Partitioning of the 2 μm circle plasmid of Saccharomyces cerevisiae: functional coordination with chromosome segregation and plasmid encoded Rep protein distribution,” J Cell Biol 149: 553-566; Velmurugan et al. (1998) “The 2 μm plasmid stability system: analyses of the interactions among plasmid- and host-encoded components.” Mol Cell Biol 18: 7466-7477. The high copy number and mitotic stability of the 2 μm plasmid is particularly advantageous in the context of the present invention, as these factors can increase expression of, e.g., polypeptides or RNAs of interest, often without imposing any significant negative effects on the host cells.

The genome of 2 μm plasmid genome encodes both a copy number control system and a partitioning system that facilitate the efficient and faithful segregation of the plasmid to daughter cells, i.e., during cell division. Faithful plasmid segregation requires the Rep1p and Rep2p proteins and a cis-acting STB locus, which is positioned near the replication origin, ORI. During replication, the 2 μm plasmid is partitioned as one entity consisting of about 3-5 closely knit plasmid foci. The extremely high stability of the plasmid in host yeast cells is a result of coupling between the plasmid segregation system and chromosome segregation. In the absence of the Rep1p and Rep2p proteins and STB DNA, plasmid and chromosome segregation are uncoupled. See, e.g., Cui et al. (2009) “The selfish yeast plasmid uses the nuclear motor Kip1p but not Cin8p for its localization and equal segregation.” J Cell Biol 185: 251-264; Mehta et al. (2002) “The 2 μm plasmid purloins the yeast cohesin complex: a mechanism for coupling plasmid partitioning and chromosome segregation?” J Cell Biol 158: 625-637, and Velmurugan et al., 2000, above. The copy number control system operates to counter missegregation events. That is, in the event of a drop in plasmid copy numbers in a daughter cell, copy number is increased by DNA amplification mediated by the plasmid encoded FLP site-specific recombinase. See, e.g., Futcher (1986) “Copy number amplification of the 2 μm circle plasmid of Saccharomyces cerevisiae,” J. Theor. Biol. 119: 197-204. Thus, the native replication and segregation control systems of the 2 μm plasmid advantageously maintain stability of the plasmid in the context of the invention.

Additional details regarding 2 μm plasmid stability can be found in Hinchliffe et al. (1994) YEAST VECTOR EP 0286424B1; Hinchliffe et al. (1997) STABLE YEAST 2 μM VECTOR U.S. Pat. No. 5,637,504; Sleep et al. 2 μM FAMILY PLASMID AND USE THEREOF US Pub. 2008/0261861; Bijvoet et al. (1991) “DNA Insertions in the Silent Regions of the 2 μm Plasmid of Saccharomyces cerevisiae Influence Plasmid Stability,” Yeast 7: 347-356; and Futcher and Cox (1984) “Copy number and the Stability of 2 μm Circle-Based Artificial Plasmids of Saccharomyces cerevisiae,” Journal of Bacteriology 157: 283-290.

Homologous recombination proceeds efficiently in yeast cells. This is particularly beneficial in the context of the present invention, e.g., to provide for homologous recombination of, e.g., a linear nucleic acid encoding a sequence of interest, with the 2 μm plasmid. For an introduction to homologous recombination, see, e.g., Muyrers et al. (2001) “Techniques: recombinogenic engineering—new options for cloning and manipulating DNA.” Trends Biochem Sci 26: 325-331. Homologous recombination has been used for the recombination of co-introduced linear expression vectors and inserts to form plasmids, as well as for the recombination of genes in vivo. See, e.g., Swers et al. (2004) “Shuffled antibody libraries created by in vivo homologous recombination and yeast surface display,” Nucleic Acids Research, 32(3) e36; 17; Mezard et al. (1992) “Recombination between similar but not identical DNA sequences during yeast transformation occurs within short stretches of identity.” Cell 70: 659-670; Abecassis et al. (2000) “High efficiency family shuffling based on multi-step PCR and in vivo DNA recombination in yeast: statistical and functional analysis of a combinatorial library between human cytochrome p450 1a1 and 1a2,” Nucl Acids Res 28: E88; and Cherry et al. (1999) “Directed evolution of a fungal peroxidase” Nat Biotech 17: 379-384. Homologous recombination between nucleic acid molecules in yeast can occur with stretches of as little as 4 nucleotides of identity (see, e.g., Schiestl and Petes (1991) “Integration of DNA fragments by illegitimate recombination in Saccharomyces cerevisiae.” Proc Natl Acad Sci USA 88: 7585-7589. However, somewhat longer stretches of sequence identity (and/or high similarity) improve the specificity and frequency of recombination. Thus, in the present invention, regions of identity/similarity are typically selected to be e.g., about 10 to about 300 or more nucleotides in length. Typical regions of similarity/identity can be in the range of about 20 to about 100 nucleotides in length, e.g., about 40 to about 75 nucleotides, e.g., about 50 to about 65 nucleotides in length. Increasing the copy number of homologous recombination sites can also increase the frequency of homologous recombination. See, e.g., Wilson et al. (1994) “The frequency of gene targeting in yeast depends on the number of target copies,” Proc Natl Acad Sci USA 91: 177-181. Accordingly, while not required, the use of multiple copies of a region of sequence identity/similarity can be used to increase homologous recombination rates.

In the subject invention, nucleic acids of interest, i.e., that are to be recombined into, e.g., a 2 μm plasmid, are generated to include regions of homology (e.g., regions with high sequence identity/similarity) with endogenous sequences present in the 2 μm plasmid. Such regions are typically in the range of 10 to 300 nucleotides in length, e.g., about 50 to 75 nucleotides in length, e.g., about 40 to 60 nucleotides in length, etc., as noted above. Upon introduction into a yeast cell comprising the 2 μm plasmid, the yeast DNA repair and recombination machinery splices portions of the nucleic acid of interest between the regions of homology into the yeast 2 μm plasmid, resulting in a recombinant 2 μm-derived plasmid comprising a region of the nucleic acid of interest.

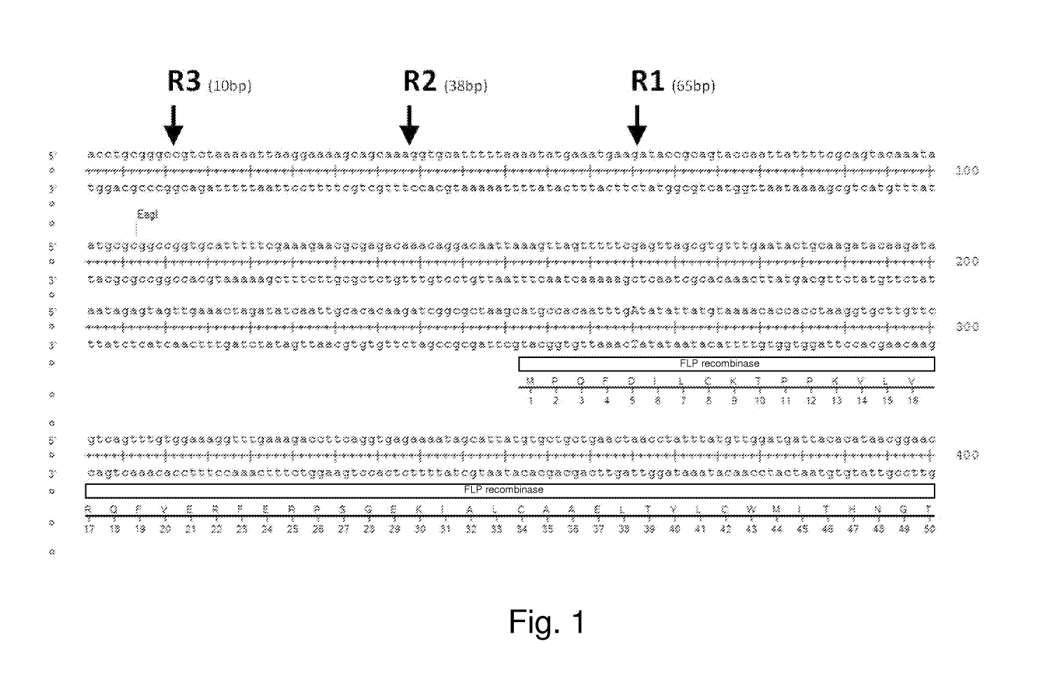

In general, homologous insertion sites are selected to minimize disruption to coding or regulatory sequences of the yeast 2 μm plasmid. Disruption of such coding or regulatory sequences can interfere with the partition or copy number control system of the plasmid, reducing stability of the plasmid during growth phase of a yeast cell culture. For example, in Sleep et al. 2 μM FAMILY PLASMID AND USE THEREOF US Patent Application Publication No. 2008/0261861 and Sleep et al. 2 μM FAMILY PLASMID AND USE THEREOF EP 1,711,602 B1, homologous insertion sites between the REP2 and FRT genes and between the FLP and FRT genes are described. One aspect of the invention is the surprising discovery that a preferred site for homologous recombination lies between the FLP and REP2 genes of the 2 μm plasmid. This finding is particularly unexpected in light of the fact that region between the FLP and REP2 genes had previously been found to be required for plasmid stability (see, e.g., U.S. Pat. No. 5,637,504 “STABLE YEAST 2 μM VECTOR” by Hinchliffe et al.). In one example, illustrated in FIGS. 1-4, and described in further detail in the Examples section herein, homologous recombination was performed to insert heterologous nucleic acids of interest comprising selectable markers (e.g., encoding hygromycin resistance) into the region between FLP and REP2 genes of a 2 μm plasmid in Saccharomyces cerevisiae.

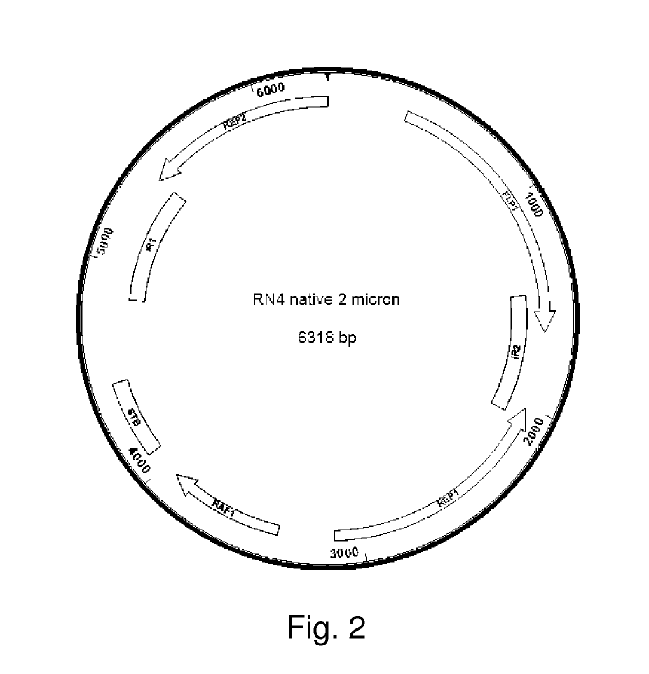

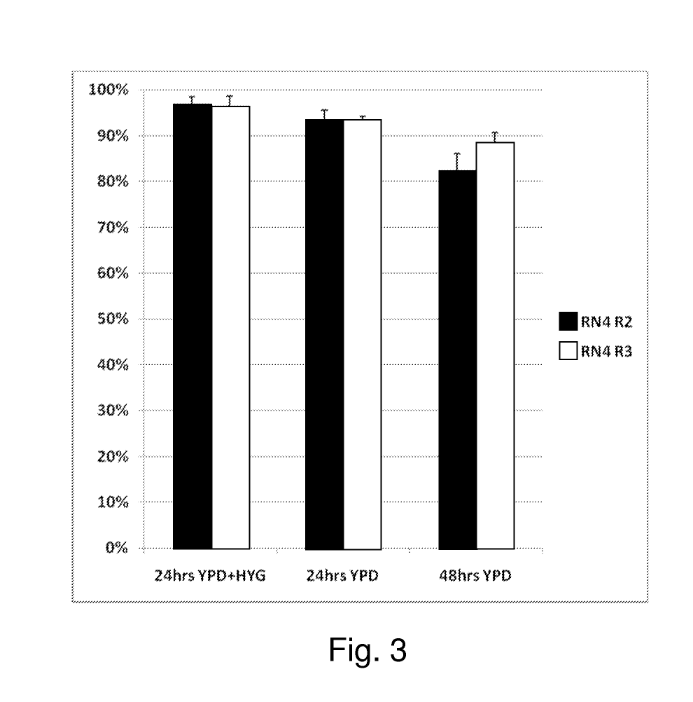

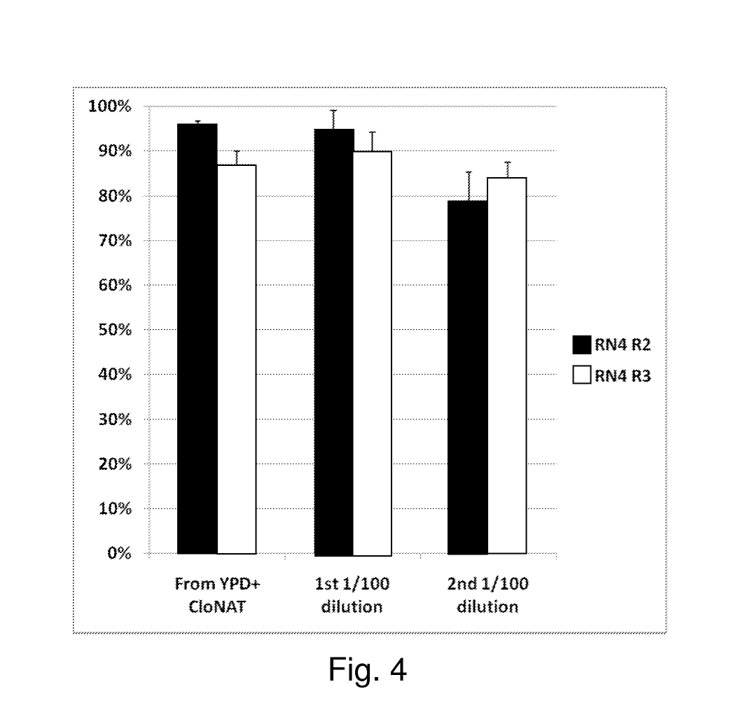

Three additional preferred insertion sites for homologous recombination include the region between REP1 and RAF1, the region between RAF1 and STB and the region between STB and IR1. These insertion sites are described in further detail in FIGS. 1 and 2 and in the examples herein. All three yielded stably recombined 2 μm plasmids, as illustrated in FIGS. 3 and 4.

### Selection in Yeast

Selection of recombinant 2 μm plasmids in yeast or other fungi can be performed according to the selectable marker that is used for selection. The nucleic acid that is introduced into yeast or fungi for recombination can include a selectable marker (e.g., a nucleic acid that encodes a selectable trait). The nucleic acid can additionally include a nucleic acid sequence of interest, e.g., a nucleic acid encoding any of polypeptide with a commercially relevant property, e.g., as noted hereinbelow.

Several basic selection methods are adaptable to the present invention. In the first, the yeast strain is auxotrophic, i.e., requires addition of an exogenous component for growth. Many such auxotrophs are known, and are routinely used for auxotrophic selection purposes. Strains that comprise the 2 μm plasmid (or that can be transformed with the plasmid) can be selected by encoding a corresponding auxotrophic marker on the introduced nucleic acid that recombines into the 2 μm plasmid.

Such auxotrophs include, for example, strains that lack an enzyme needed for production of an essential amino acid or an essential nucleic acid or nucleoside/nucleotide. The nucleic acid that recombines into the 2 μm plasmid can encode the missing enzyme, allowing yeast that comprise the introduced nucleic acid (recombined into the 2 μm plasmid) to grow in media lacking the essential amino acid or nucleic acid, etc. For example, a yeast mutant in which a gene of the uracil synthesis pathway (for example the gene encoding yeast orotidine 5′-phosphate decarboxylase) is inactivated is a uracil auxotroph. This strain is unable to synthesize uracil by itself and only grows if uracil can be taken up from the environment, or, as a selectable marker in the context of the present invention, when the orotidine 5′-phosphate decarboxylase gene is supplied via homologous recombination into the 2 μm plasmid. This is in contrast to a wild-type strain, which has an endogenous gene for orotidine 5′-phosphate decarboxylase and can grow in the absence of uracil. One advantage of auxotrophic resistance is that selective pressure is essentially continuous, as cells do not grow in unsupplemented media unless they harbor the recombinant plasmid.

A number of other useful auxotrophic strains and selectable markers can similarly be used. For example, yeast strains harboring deletion alleles of the ade2, lys2, his3, his4, trp1, leu2, and ura3 genes are available, and can be selected by incorporating the appropriate gene as a selectable marker. See also, e.g., Sikorski and Hieter (1989) “A System of Shuttle Vectors and Yeast Host Strains Designed for Efficient Manipulation of DNA in Saccharomyces cerevisiae” Genetics 122: 19-27; Barnes and Thorner (1986) “Genetic Manipulation of Saccharomyces cerevisiae by Use of the LYS2 Gene” Molecular And Cellular Biology 6: 2828-2838; and Christianson et al. (1992) “Multifunctional yeast high-copy-number shuttle vectors,” Gene, 110: 119-122. The appropriate gene is introduced into a 2 μm plasmid by homologous recombination, as noted herein, and the resulting recombinant cell is selected in minimal media lacking the relevant metabolite. For further details regarding selection in yeast see also, e.g., Ausubel (1992) Current Protocols in Molecular Biology sections 13.4.1-13.4.10 Supplement 21 (2000) “YEAST VECTORS UNIT 13.4 Yeast Cloning Vectors and Genes.”

In the second approach to selection, the introduced nucleic acid encodes an antibiotic or antifungal resistance gene, or, e.g., an antitoxin. This permits cells harboring the recombinant plasmid to survive in the presence of the antibiotic, antifungal, etc. A common marker for this purpose in yeast encodes hygromycin resistance. In the presence of hygromycin B, only cells that harbor an appropriate recombinant plasmid encoding hygromycin resistance (e.g., hygromycin B phosphotransferase) can survive. In another example, nourseothricin resistance can be used by encoding the resistance marker SAT-1 (encoding, e.g., nourseothricin N-acetyltransferase). In yet another preferred example, the marker can encode kanMX4, which permits growth in media containing G418 (also known as Geneticin®). Several other appropriate selection agents are similarly available. See also, Ausubel (1992) Current Protocols in Molecular Biology sections 13.4.1-13.4.10 Supplement 21 (2000) “YEAST VECTORS UNIT 13.4 Yeast Cloning Vectors and Genes.” To maintain selective pressure over time, the media can be supplemented at appropriate intervals with the antibiotic, antifungal or toxin. This adds to the stability of the recombinant plasmid in the culture.

A third type of selection relies on selection of an introduced trait. For example, if the introduced nucleic acid encodes a visible marker, such as a red or green florescent protein, then cells can be selected by visual inspection or automated cell sorting, e.g., via fluorescence activated cell sorting (FACS), a technique well known to those of skill in the art.

A fourth type of selection uses counter-selectable markers. These markers prevent growth in cells harboring an appropriate marker. For example, KlURA3 prevents growth in media containing 5-fluoroorotic acid; similarly, GAL1/10-p53 prevents growth in media containing galactose. As is the case with URA3, the LYS2 gene can also be selected in a positive fashion by using lysine-free medium. In this approach, the LYS2 gene encodes α-aminoadipate reductase, an enzyme that is required for lysine biosynthesis. Cells that express wild type Lys2p do not grow on media containing α-aminoadipate as a primary nitrogen source. High levels of α-aminoadipate lead to the accumulation of a toxic intermediate, while lys2 mutants do not produce of this intermediate. See also, Sikorski and Boeke (1991) “In Vitro Mutagenesis and Plasmid Shuffling: From Cloned Gene to Mutant Yeast,” in METHODS IN ENZYMOLOGY, 194: 302-318.

A fifth type of selection provides for enhanced ability to grow on an energy source present in the growth media. This can include encoding essentially any enzyme that acts in a metabolic or catabolic pathway that converts the energy source into a more readily metabolized energy source. For example, many such enzymes can be found in EC 1.1 to EC 6.6. Generally, see Enzyme Nomenclature 1992 Academic Press, San Diego, Calif., ISBN 0-12-227164-5, 0-12-227165-3, as supplemented through supplement 16 (2010).

Additional details regarding selection in yeast can be found in Wei Xiao (Editor) (2010) Yeast Protocols Humana Press ISBN-10: 1617375691, ISBN-13: 978-1617375699; Mackenzie (2006) YAC Protocols (Methods in Molecular Biology) Humana Press; 2nd edition ISBN-10: 1588296121 ISBN-13: 978-1588296122; Gellissen (Editor) (2006) Production of Recombinant Proteins: Novel Microbial and Eukaryotic Expression Systems ISBN-10: 3527310363, ISBN-13: 978-3527310364; Amberg et al. (2005) Methods in Yeast Genetics: A Cold Spring Harbor Laboratory Course Manual, Cold Spring Harbor Laboratory Press ISBN-10: 0879697288, ISBN-13: 978-0879697280; Guthrie and Fink (eds) (2002) Guide to Yeast Genetics and Molecular and Cell Biology, Part B, Volume 350 Academic Press; 1st edition ISBN-10: 0123106710, ISBN-13: 978-0123106711; Kuhla et al. (1996) “2 μm vectors containing the Saccharomyces cerevisiae metallothionein gene as a selectable marker: excellent stability in complex media, and high level expression of recombinant protein from a CUP1-promoter-controlled expression cassette in cis,” Yeast 11: 1-14.

In some cases, different forms of selection can be used in combination. For example, where the nucleic acid of interest encodes a modified enzyme of interest, an initial selectable marker can be used to select for transformed cells, and then a selective pressure appropriate to the modified enzyme can be used to select for a desired enzyme activity. Thus, for example, any of selection methods 1-5 noted above can be used to select for transformed cells, which can then have an appropriate selection method applied to select for activity of an encoded enzyme of interest.

Selection of a nucleic acid that encodes a polypeptide of interest comprising a desirable activity other than a typical selection marker is performed in an assay appropriate to the polypeptide of interest. For example, activity of an enzyme can be screened by detecting a product produced by the enzyme. Such assays are generally available, with many being described in the various references herein.

Nucleic Acid Targets for Recombination into the Yeast 2 μM Plasmid

A nucleic acid of interest can be cloned into the 2 μm plasmid, or other yeast plasmid, using the methods and compositions herein. The nucleic acid of interest can include a selectable marker and can additionally include a sequence that encodes a polypeptide or RNA of interest. This sequence can be essentially any recombinant or isolated nucleic acid that is desirably expressed in a yeast cell, e.g., a commercially valuable polypeptide or RNA. These include nucleic acids that encode polypeptides that encode enzymes, e.g., for the synthesis of polymers, biofuels, or other industrial products, as well as other biologically useful proteins, e.g., therapeutic proteins. Examples include polypeptides that catalyze or regulates degradation or synthesis of sugars, polysaccharides, cellulosic materials (e.g., cellulose, xylan, etc.), or other polymers, we well as biologically active polypeptides. Similarly, the polypeptide that is encoded can, optionally, regulate expression, synthesis, or folding of an additional polypeptide that catalyzes or regulates degradation or synthesis of a sugar, a polysaccharide, a cellulosic material, or a polymer. Examples of such regulatory polypeptides include transcription factors, polypeptides that control or regulate polypeptide or RNA turnover rates in the cell, enzymes that catalyze post-transcriptional polypeptide modifications, such as phosphorylation, prenylation, ubiquitination, or the like. Additional examples include molecular chaperones. In another example, the nucleic acid of interest optionally encodes an RNA product such as an RNAi, ribozyme, antisense, or the like, e.g., an RNA that regulates the expression of an RNA or polypeptide of interest, or an RNA that itself displays a catalytic activity of interest.

The essentially unlimited nature of the type of nucleic acids that can be incorporated into, e.g., the yeast 2 μm plasmid, makes it impractical to list all possible applications. For example, the nucleic acids of the invention can encode essentially any enzyme, e.g., those listed at EC 1.1 to EC 1.3, EC 1.4 to EC 1.97, EC 2.1 to EC 2.4.1, EC 2.4.2 to EC 2.9, EC 3.1 to EC 3.3, EC 3.4 to EC 3.13, EC 4 to EC 4.99, EC 5 to EC 5.99 and EC 6 to EC 6.6. Generally, see Enzyme Nomenclature 1992 Academic Press, San Diego, Calif., ISBN 0-12-227164-5, 0-12-227165-3, as supplemented through supplement 16 (2010). See also, e.g., Supplement 1 (1993) (Eur J Biochem 1994 223, 1-5); Supplement 2 (1994) (Eur J Biochem, 1995 232, 1-6); Supplement 3 (1995) (Eur J Biochem, 1996 237, 1-5); Supplement 4 (1997) (Eur J Biochem, 1997, 250, 1-6); Supplement 5 (1999) (Eur J Biochem, 1999, 264, 610-650); Supplement 6 (2000) (Epub only at chem.(dot)qmul(dot)ac(dot)uk/iubmb/enzyme/), Supplement 7 (2001) (id), Supplement 8 (2002) (id), Supplement 9 (2003) (id), Supplement 10 (2004) (id), Supplement 11 (2005) (id), Supplement 12 (2006) (id), Supplement 13 (2007) (id), Supplement 14 (2008) (id), Supplement 15 (2009) (id), Supplement 16 (2010) (id).

For example, just one useful application includes nucleic acids that encode enzymes that catalyze the degradation of sugars, e.g., the degradation of polysaccharides such as cellulose into fermentable sugars. This is useful e.g., for the processing of biomass, the production of biofuels, and the manufacture and degradation of food, plant products, and industrial products. Such enzymes include, e.g., the enzymes classified in the standard Nomenclature Committee of the International Union of Biochemistry and Molecular Biology (NC-IUBMB) as Enzyme Classification as 3.2.1.x. These include, for example glycosidases, e.g., enzymes hydrolysing O- and S-glycosyl compounds, including: EC 3.2.1.1 (α-amylase), EC 3.2.1.2 (β-amylase), EC 3.2.1.3 (glucan 1,4-α-glucosidase), EC 3.2.1.4 (cellulase), EC 3.2.1.6 (endo-1,3(4)-β-glucanase), EC 3.2.1.7 (inulinase), EC 3.2.1.8 (endo-1,4-β-xylanase), EC 3.2.1.10 (oligo-1,6-glucosidase), EC 3.2.1.11 (dextranase), EC 3.2.1.14 (chitinase), EC 3.2.1.15 (polygalacturonase), EC 3.2.1.17 (lysozyme), EC 3.2.1.18 (exo-α-sialidase), EC 3.2.1.20 (α-glucosidase), EC 3.2.1.21 (β-glucosidase), EC 3.2.1.22 (α-galactosidase), EC 3.2.1.23 (β-galactosidase), EC 3.2.1.24 (α-mannosidase), EC 3.2.1.25 (β-mannosidase), EC 3.2.1.26 (β-fructofuranosidase), EC 3.2.1.28 (αα-trehalase), EC 3.2.1.31 (β-glucuronidase), EC 3.2.1.32 (xylan endo-1,3-β-xylosidase), EC 3.2.1.33 (amylo-1,6-glucosidase), EC 3.2.1.35 (hyaluronoglucosaminidase), EC 3.2.1.36 (hyaluronoglucuronidase), EC 3.2.1.37 (xylan 1,4-β-xylosidase), EC 3.2.1.38 (β-D-fucosidase), EC 3.2.1.39 (glucan endo-1,3-β-D-glucosidase), EC 3.2.1.40 (β-L-rhamnosidase), EC 3.2.1.41 (pullulanase), EC 3.2.1.42 (GDP-glucosidase), EC 3.2.1.43 (β-L-rhamnosidase), EC 3.2.1.44 (fucoidanase), EC 3.2.1.45 (glucosylceramidase), EC 3.2.1.46 (galactosylceramidase), EC 3.2.1.47 (galactosylgalactosylglucosylceramidase), EC 3.2.1.48 (sucrose β-glucosidase), EC 3.2.1.49 (α-N-acetylgalactosaminidase), EC 3.2.1.50 (α-N-acetylglucosaminidase), EC 3.2.1.51 (α-L-fucosidase), EC 3.2.1.52 (β-L-N-acetylhexosaminidase), EC 3.2.1.53 (β-N-acetylgalactosaminidase), EC 3.2.1.54 (cyclomaltodextrinase), EC 3.2.1.55 (α-N-arabinofuranosidase), EC 3.2.1.56 (glucuronosyl-disulfoglucosamine glucuronidase), EC 3.2.1.57 (isopullulanase), EC 3.2.1.58 (glucan 1,3-β-glucosidase), EC 3.2.1.59 (glucan endo-1,3-α-glucosidase), EC 3.2.1.60 (glucan 1,4-α-maltotetraohydrolase), EC 3.2.1.61 (mycodextranase), EC 3.2.1.62 (glycosylceramidase), EC 3.2.1.63 (1,2-α-L-fucosidase), EC 3.2.1.64 (2,6-β-fructan 6-levanbiohydrolase), EC 3.2.1.65 (levanase), EC 3.2.1.66 (quercitrinase), EC 3.2.1.67 (galacturan 1,4-α-galacturonidase), EC 3.2.1.68 (isoamylase), EC 3.2.1.70 (glucan 1,6-α-glucosidase), EC 3.2.1.71 (glucan endo-1,2-β-glucosidase), EC 3.2.1.72 (xylan 1,3-β-xylosidase), EC 3.2.1.73 (licheninase), EC 3.2.1.74 (glucan 1,4-β-glucosidase), EC 3.2.1.75 (glucan endo-1,6-β-glucosidase), EC 3.2.1.76 (L-iduronidase), EC 3.2.1.77 (mannan 1,2-(1,3),-α-mannosidase), EC 3.2.1.78 (mannan endo-1,4-β-mannosidase), EC 3.2.1.80 (fructan β-fructosidase), EC 3.2.1.81 (agarase), EC 3.2.1.82 (exo-poly-α-galacturonosidase), EC 3.2.1.83 (κ-carrageenase), EC 3.2.1.84 (glucan 1,3-β-glucosidase), EC 3.2.1.85 (6-phospho-β-galactosidase), EC 3.2.1.86 (6-phospho-α-glucosidase), EC 3.2.1.87 (capsular-polysaccharide endo-1,3-α-galactosidase), EC 3.2.1.88 (β-L-arabinosidase), EC 3.2.1.89 (arabinogalactan endo-1,4-β-galactosidase), EC 3.2.1.91 (cellulose 1,4-(3-cellobiosidase), EC 3.2.1.92 (peptidoglycan β-N-acetylmuramidase), EC 3.2.1.93 (α-phosphotrehalase), EC 3.2.1.94 (glucan 1,6-α-isomaltosidase), EC 3.2.1.95 (dextran 1,6-α-isomaltotriosidase), EC 3.2.1.96 (mannosyl-glycoprotein endo-β-N-acetylglucosaminidase), EC 3.2.1.97 (glycopeptide α-N-acetylgalactosaminidase), EC 3.2.1.98 (glucan 1,4-α-maltohexaosidase), EC 3.2.1.99 (arabinan endo-1,5-α-L-arabinosidase), EC 3.2.1.100 (mannan 1,4-mannobiosidase), EC 3.2.1.101 (mannan endo-1,6-α-mannosidase), EC 3.2.1.102 (blood-group-substance endo-1,4-β-galactosidase), EC 3.2.1.103 (keratan-sulfate endo-1,4-β-galactosidase), EC 3.2.1.104 (steryl-β-glucosidase), EC 3.2.1.105 (strictosidine β-glucosidase), EC 3.2.1.106 (mannosyl-oligosaccharide glucosidase), EC 3.2.1.107 (protein-glucosylgalactosylhydroxylysine glucosidase), EC 3.2.1.108 (lactase), EC 3.2.1.109 (endogalactosaminidase), EC 3.2.1.110 (mucinaminylserine mucinaminidase), EC 3.2.1.111 (1,3-α-L-fucosidase), EC 3.2.1.112 2-(deoxyglucosidase), EC 3.2.1.113 (mannosyl-oligosaccharide 1,2-α-mannosidase), EC 3.2.1.114 (mannosyl-oligosaccharide 1,3-1,6-α-mannosidase), EC 3.2.1.115 (branched-dextran exo-1,2-α-glucosidase), EC 3.2.1.116 (glucan 1,4-α-maltotriohydrolase), EC 3.2.1.117 (amygdalin β-glucosidase), EC 3.2.1.118 (prunasin β-glucosidase), EC 3.2.1.119 (vicianinβ-glucosidase), EC 3.2.1.120 (oligoxyloglucan β-glycosidase), EC 3.2.1.121 (polymannuronate hydrolase), EC 3.2.1.122 (maltose-6′-phosphate glucosidase), EC 3.2.1.123 (endoglycosylceramidase), EC 3.2.1.124 (3-deoxy-2-octulosonidase) EC 3.2.1.125 (raucaffricine β-glucosidase) EC 3.2.1.126 (coniferin β-glucosidase), EC 3.2.1.127 (1,6-α-L-fucosidase), EC 3.2.1.128 (glycyrrhizinate β-glucuronidase), EC 3.2.1.129 (endo-α-sialidase), EC 3.2.1.130 (glycoprotein endo-α-1,2-mannosidase), EC 3.2.1.131 (xylan α-1,2-glucuronosidase), EC 3.2.1.132 (chitosanase), EC 3.2.1.133 (glucan 1,4-α-maltohydrolase), EC 3.2.1.134 (difructose-anhydride synthase), EC 3.2.1.135 (neopullulanase) EC 3.2.1.136 (glucuronoarabinoxylan endo-1,4-(3-xylanase), EC 3.2.1.137 (mannan exo-1,2-1,6-β-mannosidase), EC 3.2.1.139 (α-glucuronidase), EC 3.2.1.140 (lacto-N-biosidase), EC 3.2.1.141 (4-α-D-{(1→4)-α-D-glucano}trehalose trehalohydrolase) EC 3.2.1.142 (limit dextrinase), EC 3.2.1.143 (poly(ADP-ribose) glycohydrolase), EC 3.2.1.144 (β-deoxyoctulosonase), EC 3.2.1.145 (galactan 1,3-β-galactosidase), EC 3.2.1.146 (β-galactofuranosidase), EC 3.2.1.147 (thioglucosidase), EC 3.2.1.149 (β-primeverosidase), EC 3.2.1.150 (oligoxyloglucan reducing-end-specific cellobiohydrolase), EC 3.2.1.151 (xyloglucan-specific endo-β-1,4-glucanase), EC 3.2.1.152 (mannosylglycoprotein endo-β-mannosidase), EC 3.2.1.153 (fructan β-(2,1)-fructosidase), EC 3.2.1.154 (fructan β-(2,6)-fructosidase), EC 3.2.1.156 (oligosaccharide reducing-end xylanase), EC 3.2.1.157 (l-carrageenase); EC 3.2.1.158 (α-agarase), EC 3.2.1.159 (α-neoagaro-oligosaccharide hydrolase), EC 3.2.1.161 (β-apiosyl-β-glucosidase), EC 3.2.1.162 (λ-carrageenase), EC 3.2.1.163 (1,6-α-D-mannosidase), EC 3.2.1.164 (galactan endo-1,6-β-galactosidase), and EC 3.2.1.165 (exo-1,4-β-D-glucosaminidase).

Other useful enzymes with glycosylase activity, which can be encoded by the nucleic acids of the invention, include those listed at EC 3.2.2.x (glycosylases that hydrolyse N-Glycosyl Compounds) and EC 3.2.1.147 (thioglucosidase).

In particularly preferred embodiments, a nucleic acid of interest that can be cloned into the 2 μm plasmid, or other yeast plasmid, includes a sequence that encodes a dehydrogenase (EC 1.1.1-EC1.21.1.1 and EC 1.97.1.1-EC 1.97.1.12); a dehydratase (EC 4.2.1-EC 4.2.1.129), or an invertase (EC 3.2.1.26).

A dehydrogenase is an enzyme that oxidises a substrate by a reduction reaction that transfers one or more hydrides (H—) to an electron acceptor, usually NAD⁺/NADP⁺ or a flavin coenzyme such as FAD or FMN. Dehydrogenases are present in a wide variety of organisms, and play central roles in, e.g., energy metabolism, aerobic respiration, cell development, genetic disease, etc. Numerous dehydrogenases are known in the art. For example, aldehyde dehydrogenases catalyze the oxidation (i.e., dehydrogenation) of aldehydes via the mechanism below:

R—CHO+NAD+H₂O→R—COOH+NADH+H⁺

Acetaldehyde dehydrogenases are dehydrogenase enzymes that catalyze the conversion of acetaldehyde into acetic acid in an oxidation reaction that can be generally summarized as follows:

CH₃CHO+NAD⁺+CoA→acetyl-CoA+NADH+H⁺

Alcohol dehydrogenases (ADH) catalyze the interconversion between alcohols and aldehydes or ketones with the reduction of nicotinamide adenine dinucleotide (NAD⁺ to NADH). Glutamate dehydrogenases that converts glutamate to α-Ketoglutarate, and vice versa. Lactate dehydrogenases catalyzes the interconversion of pyruvate and lactate with concomitant interconversion of NADH and NAD⁺. Further information regarding dehydrogenase enzymes can be found, e.g., at the Aldehyde Dehydrogenase Gene Superfamily Database, i.e., a publicly available database on the World Wide Web (www(dot)aldh(dot)org/overview(dot)php); the enzyme nomenclature database on the World Wide Web (www(dot)chem(dot)qmul(dot)ac(dot)uk/iubmb/enzyme/); and Toseland et al. (2005) “DSD—An integrated, web-accessible database of Dehydrogenase Enzyme Stereospecificities.” BMC Bioinformatics 6: 283-289.

A dehydratase is an enzyme that catalyzes the removal of oxygen and hydrogen from organic compounds in the form of water, i.e., in a process also known as dehydration. There are four classes of dehydratases: dehydratases that act on 3-hydroxyacyl-CoA esters and do not use cofactors; [4Fe-4S]-containing dehydratases that act on 2-hydroxyacyl-CoA esters (radical reaction, [4Fe-4S] cluster containing) and require reductive activation by an ATP-dependent one-electron transfer; [4Fe-45]- and FAD-containing dehydratases that act on 4-hydroxyacyl-CoA esters; and dehydratases that contain an [4Fe-4S] cluster as active site (e.g., aconitase, fumarase, serine dehydratase, etc.). Further information regarding these enzymes can be found in, e.g., Lewis et al. (2011) “Enzymatic Functionalization of Caron-Hydrogen Bonds.” Chem Soc Rev 40: 2003-21; and the enzyme nomenclature database on the World Wide Web (www(dot)chem(dot)qmul(dot)ac(dot)uk/iubmb/enzyme/).

An invertase is an enzyme that catalyzes the hydrolysis of sucrose to produce inverted sugar syrup, i.e., a mixture of fructose and glucose. Invertase plays a central role in ethanol fermentation and can be used to convert lignocellulosic material into ethanol, e.g., for use as a solvent, germicide, antifreezer, etc. Further information regarding invertases can be found in, e.g., Roitsch, et al. (2004) “Function and regulation of plant invertases: sweet sensations.” Trends Plant Sci 9: 606-613; Ruan et al. (2010) “Sugar input, metabolism, and signaling mediated by invertase: roles in development, yield potential, and response to drought and heat.” Mol Plant 3: 942-955; del Castillo Agudo, et al. (1994) “Genes involved in the regulation of invertase production in Saccharomyces cerevisiae.” Microbiologia 10: 385-394; and the enzyme nomenclature database on the World Wide Web (www(dot)chem(dot)qmul(dot)ac(dot)uk/iubmb/enzyme/).

Similarly, there is an ever growing set of biologically active, therapeutic and/or diagnostic polypeptides that can be encoded by the nucleic acids of the invention. These include, but are not limited to, e.g., a variety of fluorescent and luminescent proteins such as green and red fluorescent proteins, acylases, acyltransferases, aldoses, an aldosterone receptor, amidases, an antibody, an antibody fragment, α-1 antitrypsin, angiostatin, antihemolytic factor, apolipoprotein, apoprotein, atrial natriuretic factor, atrial natriuretic polypeptide, atrial peptide, a C—X—C chemokine, T39765, NAP-2, ENA-78, Gro-α, Gro-β, Gro-γ, IP-10, GCP-2, NAP-4, SDF-1, PF4, MIG, calcitonin, c-kit ligand, a cytokine, a CC chemokine, a corticosterone, estrogen receptor, Met, methyl-transferases, monocyte chemoattractant protein-1, monocyte chemoattractant protein-2, monocyte chemoattractant protein-3, monocyte inflammatory protein-1α, monocyte inflammatory protein-1β, monooxygenase, Mos, Myc, RANTES, I309, R83915, R91733, HCC1, T58847, D31065, T64262, CD40, CD40 ligand, CD44, c-kit ligand, collagen, colony stimulating factor (CSF), complement factor 5a, complement inhibitor, complement receptor 1, epithelial neutrophil activating peptide-78, MGSA, MIP1-α, MIP1-β, MIP1-δ, enone reductases, epidermal growth factor (EGF), epithelial neutrophil activating peptide, erythropoietin (EPO), exfoliating toxin, dehalogenases, Factor IX, Factor VII, Factor VIII, Factor X, fibroblast growth gactor (FGF), fibrinogen, fibronectin, Fos, G-CSF, GM-CSF, glucocerebrosidase, gonadotropin, growth factor, growth factor receptor, hyalurin, hedgehog protein, hemoglobin, hepatocyte growth gactor (HGF), hirudin, human serum albumin, ICAM-1, an ICAM-1 receptor, an LFA-1, LFA-1 receptor, an inflammatory protein, insulin, insulin-like Growth Factor (IGF), IGF-I, IGF-II, interferon, IFN-α, IFN-β, IFN-γ, interleukin, IL-1, IL-2, IL-3, IL-4, IL-5, IL-6, IL-7, IL-8, IL-9, IL-10, IL-11, IL-12, Jun, keratinocyte growth factor (KGF), ketoreductases, lactoferrin, leukemia inhibitory factor, LDL receptor, luciferase, Myb, neurturin, neutrophil inhibitory factor (NIF), nitrilases, oncostatin M, osteogenic protein, oncogene product, oxidases, parathyroid hormone, PD-ECSF, PDGF, peptide hormone, progesterone receptor, human growth hormone, p53, pleiotropin, Protein A, Protein G, pyrogenic exotoxin A, B, or C, Ras, Raf, Rel, relaxin, renin, a signal transduction protein, SCF/c-kit, Soluble complement receptor I, Soluble I-CAM 1, Soluble interleukin receptor, Soluble TNF receptor, Somatomedin, Somatostatin, Somatotropin, Streptokinase, Superantigen, Staphylococcal enterotoxin, SEA, SEB, SEC1, SEC2, SEC3, SED, SEE, steroid hormone receptor, Superoxide dismutase, Tat, Testosterone Receptor, Toxic shock syndrome toxin, Thymosin alpha 1, Tissue plasminogen activator, tumor growth factor (TGF), TGF-α variants, TGF-13, Transaminases, a transcriptional activator protein, a transcriptional suppressor protein, Tumor Necrosis Factor, Tumor Necrosis Factor cc, Tumor necrosis factor 13, Urokinase, VLA-4 protein, VCAM-1 protein, Vascular Endothelial Growth Factor (VEGEF), and many others. Preferred targets for expression in yeast can include any of those already noted, including e.g., ketoreductases, transaminases, enone reductases, dehydrogenases, dehalogenases, nitrilases, monooxygenase, methyl-transferases, and oxidases.

Mutations, Combinatorial Libraries and Other Applications

In addition to expressing available polypeptides, genes of interest can be mutated, e.g., by various combinatorial shuffling or other available mutagenesis procedures, and cloned into yeast or other fungi using homologous recombination as noted herein. In one useful application, combinatorial libraries of homologous nucleic acids, e.g., encoding variants of the polypeptides noted above, are generated and screened for activity.

In such applications, new or improved polypeptides and/or RNAs, or a polynucleotide encoding a reference polypeptide, such as a wild type enzyme, can be subjected to mutagenesis to produce a library of variant polynucleotides encoding polypeptide variants that display changes in amino acid sequence, relative to a wild type polypeptide or RNA. Screening of the variants for a desired property, such as an improvement in enzyme activity or stability, modified regulation or expression, improved or reduced translation, activity against new substrates, or the like, allows for the identification of amino acid residues associated with the desired property. For a review of directed evolution and mutation approaches see, e.g., Turner (2009) “Directed evolution drives the next generation of biocatalysts” Nat Chem Biol 5: 567-573; Fox and Huisman (2008), “Enzyme optimization: moving from blind evolution to statistical exploration of sequence-function space,” Trends Biotechnol 26: 132-138; Arndt and Miller (2007) Methods in Molecular Biology, Vol. 352: Protein Engineering Protocols, Humana; Zhao (2006) Comb Chem High Throughput Screening 9: 247-257; Bershtein et al. (2006) Nature 444: 929-932; Brakmann and Schwienhorst (2004) Evolutionary Methods in Biotechnology: Clever Tricks for Directed Evolution, Wiley-VCH, Weinheim; and Rubin-Pitel Arnold and Georgiou (2003) Directed Enzyme Evolution: Screening and Selection Methods, 230, Humana, Totowa. For example, nucleic acid shuffling (in vitro, in vivo, and/or in silico) has been used in a variety of ways, e.g., in combination with homology-, structure-, or sequence-based analysis and with a variety of recombination or selection protocols a variety of methods. See, e.g., WO/2000/042561 by Crameri et al. OLIGONUCLEOTIDE MEDIATED NUCLEIC ACID RECOMBINATION; WO/2000/042560 by Selifonov et al. METHODS FOR MAKING CHARACTER STRINGS, POLYNUCLEOTIDES AND POLYPEPTIDES; WO/2001/075767 by GUSTAFSSON et al. 1N SILICO CROSS-OVER SITE SELECTION; and WO/2000/004190 by del Cardayre EVOLUTION OF WHOLE CELLS AND ORGANISMS BY RECURSIVE SEQUENCE RECOMBINATION.

In one preferred combinatorial library approach, individual sites of a polypeptide of interest are varied, either randomly or according to a logical rule or filter (e.g., by taking structure or various heuristic filtering procedures into account). Nucleic acids encoding such variant polypeptides are constructed by PCR-based reassembly, e.g., splicing by overlap extension PCR (“SOE PCR”). Examples of such methods are descried in U.S. Ser. No. 61/283,877 filed Dec. 9, 2009, entitled REDUCED CODON MUTAGENESIS by Fox et al.; U.S. Ser. No. 61/061,581 filed Jun. 13, 2008 entitled METHOD OF SYNTHESIZING POLYNUCLEOTIDE VARIANTS by Colbeck et al.; U.S. Ser. No. 12/483,089 filed Jun. 11, 2009 entitled METHOD OF SYNTHESIZING POLYNUCLEOTIDE VARIANTS by Colbeck et al.; PCT/US2009/047046 filed Jun. 11, 2009 entitled METHOD OF SYNTHESIZING POLYNUCLEOTIDE VARIANTS by Colbeck et al.; U.S. Ser. No. 12/562,988 filed Sep. 18, 2009 entitled COMBINED AUTOMATED PARALLEL SYNTHESIS OF POLYNUCLEOTIDE VARIANTS by Colbeck et al.; and PCT/US2009/057507 filed Sep. 18, 2009, entitled COMBINED AUTOMATED PARALLEL SYNTHESIS OF POLYNUCLEOTIDE VARIANTS by Colbeck et al., all incorporated herein by reference. These procedures include “Automated Parallel SOEing” (“APS”), or “Multiplexed Gene SOEing,” which use a variety of PCR-reassembly methods, including SOE-PCR, e.g., in automated or automatable formats. Further details regarding splicing by overlap extension methods can also be found in Horton et al. (1989) “Engineering hybrid genes without the use of restriction enzymes: gene splicing by overlap extension,” Gene 77: 61-68; Horton et al. (1990) “Gene splicing by overlap extension: tailor-made genes using the polymerase chain reaction” Biotechniques 8: 528-535; Horton et al. (1997) “Splicing by overlap extension by PCR using asymmetric amplification: an improved technique for the generation of hybrid proteins of immunological interest” Gene 186: 29-35, and in PCR Cloning Protocols (Methods in Molecular Biology) Bing-Yuan Chen (Editor), Harry W. Janes (Editor) Humana Press; 2nd edition (2002) ISBN-10: 0896039692, all incorporated herein by reference.

In general, any of a variety of site saturation and other mutagenesis methods can be used for nucleic acid construction, e.g., by incorporating oligonucleotides comprising a desired variant during nucleic acid construction in the relevant assembly method. Approaches that can be adapted to the invention include those in Fox and Huisman (2008), Trends Biotechnol 26: 132-138; Arndt and Miller (2007) Methods in Molecular Biology, Vol. 352: Protein Engineering Protocols, Humana; Zhao (2006) Comb Chem High Throughput Screening 9: 247-257; Bershtein et al. (2006) Nature 444: 929-932; Brakmann and Schwienhorst (2004) Evolutionary Methods in Biotechnology: Clever Tricks for Directed Evolution, Wiley-VCH, Weinheim; and Rubin-Pitel Arnold and Georgiou (2003) Directed Enzyme Evolution: Screening and Selection Methods, 230, Humana, Totowa; as well as those in, e.g., Rajpal et al. (2005) “A General Method for Greatly Improving the Affinity of Antibodies Using Combinatorial Libraries.” Proc Natl Acad Sci USA 102: 8466-8471; Reetz et al. (2008) “Addressing the Numbers Problem in Directed Evolution” ChemBioChem 9: 1797-1804 and Reetz et al. (2006) “Iterative Saturation Mutagenesis on the Basis of B Factors as a Strategy for Increasing Protein Thermostability” Angew Chem 118: 7907-7915), all incorporated herein by reference.

Additional information on mutation formats for production of variants to be cloned into the relevant plasmid, e.g., a 2 μm plasmid, and expressed in yeast is found in Sambrook 2001 and Ausubel, herein, as well as in In Vitro Mutagenesis Protocols (Methods in Molecular Biology) Jeff Braman (Editor) Humana Press; 2nd edition (2002) ISBN-10: 0896039102; Chromosomal Mutagenesis (Methods in Molecular Biology) Gregory D. Davis (Editor), Kevin J. Kayser (Editor) Humana Press; 1st edition (2007) ISBN-10: 158829899X; PCR Cloning Protocols (Methods in Molecular Biology) Bing-Yuan Chen (Editor), Harry W. Janes (Editor) Humana Press; 2nd edition (2002) ISBN-10: 0896039692; Directed Enzyme Evolution: Screening and Selection Methods (Methods in Molecular Biology) Frances H. Arnold (Editor), George Georgiou (Editor) Humana Press; 1st edition (2003) ISBN-10: 58829286X; Directed Evolution Library Creation: Methods and Protocols (Methods in Molecular Biology) (Hardcover) Frances H. Arnold (Editor), George Georgiou (Editor) Humana Press; st1 edition (2003) ISBN-10: 1588292851; Short Protocols in Molecular Biology (2 volume set); Ausubel et al. (Editors) Current Protocols; 52 edition (2002) ISBN-10: 0471250929; and PCR Protocols A Guide to Methods and Applications (Innis et al. eds) Academic Press Inc. San Diego, Calif. (1990) (Innis).

The following publications and references provide additional detail on various available mutation formats that can be used to produce a nucleic acid of interest that can be used for homologous recombination into a yeast or other fungal plasmid, e.g., the yeast 2 μm plasmid: Arnold (1993) “Protein engineering for unusual environments,” Current Opinion in Biotechnology 4: 450-455; Bass et al. (1988) “Mutant Trp repressors with new DNA-binding specificities,” Science 242: 240-245; Botstein & Shortle (1985) “Strategies and applications of in vitro mutagenesis,” Science 229: 1193-1201; Carter et al. (1985) “Improved oligonucleotide site-directed mutagenesis using M13 vectors,” Nucl Acids Res 13: 4431-4443; Carter (1986) “Site-directed mutagenesis,” Biochem J 237: 1-7; Carter (1987) “Improved oligonucleotide-directed mutagenesis using M13 vectors,” Methods in Enzymol 154: 382-403; Dale et al. (1996) “Oligonucleotide-directed random mutagenesis using the phosphorothioate method,” Methods Mol Biol 57: 369-374; Eghtedarzadeh & Henikoff (1986) “Use of oligonucleotides to generate large deletions,” Nucl Acids Res 14: 5115; Fritz et al. (1988) “Oligonucleotide-directed construction of mutations: a gapped duplex DNA procedure without enzymatic reactions in vitro,” Nucl Acids Res 16: 6987-6999; Grundström et al. (1985) “Oligonucleotide-directed mutagenesis by microscale ‘shot-gun’ gene synthesis,” Nucl Acids Res 13: 3305-3316; Kunkel, “The efficiency of oligonucleotide directed mutagenesis,” in Nucleic Acids & Molecular Biology (Eckstein, F. and Lilley, D. M. J. eds., Springer Verlag, Berlin)) (1987); Kunkel (1985) “Rapid and efficient site-specific mutagenesis without phenotypic selection,” Proc Natl Acad Sci USA 82: 488-492; Kunkel et al. (1987) “Rapid and efficient site-specific mutagenesis without phenotypic selection,” Methods in Enzymol 154: 367-382; Kramer et al. (1984) “The gapped duplex DNA approach to oligonucleotide-directed mutation construction,” Nucl Acids Res 12: 9441-9456; Kramer & Fritz (1987) “Oligonucleotide-directed construction of mutations via gapped duplex DNA,” Methods in Enzymol 154: 350-367; Kramer et al. (1984) “Point Mismatch Repair,” Cell 38: 879-887; Kramer et al. (1988) “Improved enzymatic in vitro reactions in the gapped duplex DNA approach to oligonucleotide-directed construction of mutations,” Nucl Acids Res 16: 7207; Ling et al. (1997) “Approaches to DNA mutagenesis: an overview,” Anal Biochem 254: 157-178; Lorimer and Pastan (1995) Nucl Acids Res 23: 3067-3068; Mandecki (1986) “Oligonucleotide-directed double-strand break repair in plasmids of Escherichia coli: a method for site-specific mutagenesis,” Proc Natl Acad Sci USA 83: 7177-7181; Nakamaye & Eckstein (1986) “Inhibition of restriction endonuclease Nci I cleavage by phosphorothioate groups and its application to oligonucleotide-directed mutagenesis,” Nucl Acids Res 14: 9679-9698; Nambiar et al. (1984) “Total synthesis and cloning of a gene coding for the ribonuclease S protein,” Science 223: 1299-1301; Sakamar and Khorana (1984) “Total synthesis and expression of a gene for the a-subunit of bovine rod outer segment guanine nucleotide-binding protein (transducin),” Nucl Acids Res 14: 6361-6372; Sayers et al. (1988) “Y-T Exonucleases in phosphorothioate-based oligonucleotide-directed mutagenesis,” Nucl Acids Res 16: 791-802; Sayers et al. (1988) “Strand specific cleavage of phosphorothioate-containing DNA by reaction with restriction endonucleases in the presence of ethidium bromide,” Nucl Acids Res 16: 803-814; Sieber, et al. (2001) Nature Biotech 19: 456-460; Smith (1985) “In vitro mutagenesis,” Ann. Rev. Genet. 19: 423-462; Zoller and Smith (1983) Methods in Enzymol 100: 468-500; Zoller and Smith (1987) Methods in Enzymol. 154: 329-350; Stemmer (1994) Nature 370: 389-391; Taylor et al. (1985) “The use of phosphorothioate-modified DNA in restriction enzyme reactions to prepare nicked DNA,” Nucl Acids Res 13: 8749-8764; Taylor et al. (1985) “The rapid generation of oligonucleotide-directed mutations at high frequency using phosphorothioate-modified DNA,” Nucl Acids Res 13: 8765-8787; Wells et al. (1986) “Importance of hydrogen-bond formation in stabilizing the transition state of subtilisin,” Phil Trans R Soc Lond A 317: 415-423; Wells et al. (1985) “Cassette mutagenesis: an efficient method for generation of multiple mutations at defined sites,” Gene 34: 315-323; and Zoller & Smith (1982) “Oligonucleotide-directed mutagenesis using M13-derived vectors: an efficient and general procedure for the production of point mutations in any DNA fragment,” Nucl Acids Res 10: 6487-6500. Additional details on many of the above methods can be found in Methods Enzymol Volume 154, which also describes various controls for trouble-shooting problems with several mutagenesis methods. All of the foregoing references are incorporated herein by reference.

In several formats, polynucleotides encoding polypeptides with a defined amino acid sequence permutation are generated. For example, a set of amplicons comprising the permutations and having complementary overlapping regions can be selected and assembled under conditions that permit annealing of the complementary overlapping regions to each other. For example, the amplicons can be denatured and then allowed to anneal to form a complex of amplicons that together encode the polypeptide with a defined amino acid sequence permutation having one or more of the amino acid residue differences relative to a reference sequence. Generally, assembly of each set of amplicons can be carried out separately such that the polynucleotide encoding one amino acid sequence permutation is readily distinguished from another polynucleotide encoding a different amino acid sequence permutation. In some embodiments the assembly can be carried out in addressable locations on a substrate (e.g., an array) such that a plurality of polynucleotides encoding a plurality of defined amino acid sequence permutations can be generated simultaneously.

In the present invention, amplification primers can be designed to either include or amplify the relevant homologous sequence from the 2 μm plasmid, as well as any nucleic acid sequences of interest (including, e.g., a polypeptide or an RNA, a selectable marker, etc.). These sequences are then spliced into the relevant PCR or other amplification product, e.g., by overlap extension as noted above. In direct synthesis approaches, nucleic acids are synthesized to comprise the relevant homologous recombination and other sequences. In ligation approaches, the homologous sequences can be assembled with heterologous nucleic acid sequences of interest and/or nucleic acids that encode a selectable marker via ligation.

Generally, amplification to produce variant nucleic acids that can be recombined into the 2 μm plasmid as noted herein can use any enzyme used for polymerase mediated extension reactions, such as Taq polymerase, Pfu polymerase, Pwo polymerase, Tfl polymerase, rTth polymerase, Tli polymerase, Tma polymerases, or a Klenow fragment. Conditions for amplifying a polynucleotide segment using polymerase chain reaction can follow standard conditions known in the art. See, e.g., Viljoen, et al. (2005) Molecular Diagnostic PCR Handbook Springer, ISBN 1402034032; PCR Cloning Protocols (Methods in Molecular Biology) Bing-Yuan Chen (Editor), Harry W. Janes (Editor) Humana Press; 2nd edition (2002) ISBN-10: 0896039692; Directed Enzyme Evolution: Screening and Selection Methods (Methods in Molecular Biology) Frances H. Arnold (Editor), George Georgiou (Editor) Humana Press; 1st edition (2003) ISBN-10: 58829286X; Directed Evolution Library Creation: Methods and Protocols (Methods in Molecular Biology) (Hardcover) Frances H. Arnold (Editor), George Georgiou (Editor) Humana Press; st1 edition (2003) ISBN-10: 1588292851; Short Protocols in Molecular Biology (2 volume set); Ausubel et al. (Editors) Current Protocols; 52 edition (2002) ISBN-10: 0471250929; and PCR Protocols A Guide to Methods and Applications (Innis et al. eds.) Academic Press Inc. San Diego, Calif. (1990) (Innis), all incorporated herein by reference.

As noted, in addition to PCR-based methods, the 2 μm homologous recombination sequences can be spliced to heterologous nucleic acid sequences of interest by any of a variety of methods, including direct gene synthesis (e.g., sequences for the nucleic acids are recombined in silico and the resulting sequence is synthesized on a commercially available gene synthesis machine), or via ligase mediated methods such as ligation and/or the ligase chain reaction (LCR). Sequences of interest can also be assembled via standard cloning methodologies. Available cloning methods are described in a variety of standard references, e.g., Principles and Techniques of Biochemistry and Molecular Biology Wilson and Walker (Editors), Cambridge University Press 6th edition (2005) ISBN-10: 0521535816; Sambrook et al., Molecular Cloning—A Laboratory Manual (3rd Ed.), Vol. 1-3, Cold Spring Harbor Laboratory, Cold Spring Harbor, N.Y., 2001 (“Sambrook I”); The Condensed Protocols from Molecular Cloning: A Laboratory Manual Joseph Sambrook Cold Spring Harbor Laboratory Press; 1st edition (2006) ISBN-10: 0879697717 (“Sambrook I”); Current Protocols in Molecular Biology, F. M. Ausubel et al., eds., Current Protocols, a joint venture between Greene Publishing Associates, Inc. and John Wiley & Sons, Inc., (“Ausubel I”); Short Protocols in Molecular Biology Ausubel et al. (Editors) Current Protocols; 52 edition (2002) ISBN-10: 0471250929 (Ausubel II); Lab Ref, Volume 1: A Handbook of Recipes, Reagents, and Other Reference Tools for Use at the Bench Jane Roskams (Author), Linda Rodgers (Author) Cold Spring Harbor Laboratory Press (2002) ISBN-10: 0879696303; and Berger and Kimmel, Guide to Molecular Cloning Techniques, Methods in Enzymology volume 152 Academic Press, Inc., San Diego, Calif. (Berger)).

After or concurrent with nucleic acid construction, it can be desirable to pool polynucleotide variants for cloning and/or screening. However, this is not required in all cases. In some embodiments, polynucleotide variants can be assembled into an addressable library, e.g., with each address encoding a different variant polypeptide having a defined amino acid residue difference. This addressable library, e.g., of clones can be transformed into yeast or other fungal cells as noted herein, e.g., for translation and, optionally, automated plating and picking of colonies. Sequencing can be carried out to confirm mutations or combinations of mutations in each variant polypeptide sequence of the resulting transformed addressable library. Assays of the variant polypeptides for desired altered traits can be carried out on all of the variant polypeptides, or optionally on only those variant polypeptides confirmed by sequencing as having a desired mutation or combination of mutations.

In many approaches, however, nucleic acids are pooled. A pooled library of assembled nucleic acids can be transformed into yeast or other fungal cells for homologous recombination, expression, plating, picking of colonies, etc. Assay of colonies from this pooled library of clones can be carried out (e.g., via high-throughput screening) before sequencing to identify polynucleotide variants encoding polypeptides having desired altered traits. Once such a “hit” for an altered trait is identified, it can be sequenced to determine the specific combination of mutations present in the polynucleotide variant sequence. Optionally, those variants encoding polypeptides not having the desired altered traits sought in assay need not be sequenced. Accordingly, the pooled library of clones method can provide more efficiency by requiring only a single transformation rather than a set of parallel transformation reactions; screening is also simplified, as a combined library can be screened without the need to keep separate library members at separate addresses.

Pooling can be performed in any of several ways. Variants can, optionally, be pooled prior to introduction into yeast, with the homologous recombination steps being performed on pooled materials. In some protocols as noted above, this approach is not optimal, e.g., in simultaneous amplification and cloning (e.g., cloning without use of restriction sites, e.g., PCR with variant primers on circular templates), because PCR products tend to concatenate. In these and other cases, variants can be pooled after being cloned into a vector of interest, e.g., prior to transformation.

### Sequence Comparison, Identity, and Homology

New yeast plasmids are a feature of the invention. The present invention also provides variants of such plasmids, e.g., plasmids that comprise particular residues (e.g., those unique to RN4, as compared to A364A), as well as variants that comprise regions of identity with the new plasmids. The terms “identical” or “percent identity,” in the context of two or more nucleic acid or polypeptide sequences, e.g., two plasmids, refers to two or more sequences or subsequences that are the same or have a specified percentage of amino acid residues or nucleotides that are the same, when compared and aligned for maximum correspondence, as measured using one of the sequence comparison algorithms described below (or other algorithms available to persons of skill) or by visual inspection. In one aspect, the present invention relates to nucleic acid plasmids that are at least about 75%, 85%, 90%, 95%, 99%, 99.5%, or 99.8% identical to those of the sequence listings herein, or that comprise sequences of at least 100, 500, or 1,000 or more contiguous nucleotides that display 75%, 85%, 90%, 95%, 99%, 99.5%, or 99.8% identity when aligned for maximum alignment. For example, a plasmid that can be used in the compositions and methods of the invention can comprises a subsequence that is at least 90%, at least 91%, at least 92%, at least 93%, at least 94%, at least 95%, at least 96%, at least 97%, at least 98%, or at least 99% identical to a full-length endogenous 2 μm plasmid sequence from yeast RN4 or A364A (SEQ ID NO: 1; GeneBank J01347.1).

For sequence comparison and homology determination, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

One example of an algorithm that is suitable for determining percent sequence identity and sequence similarity is the BLAST algorithm, which is described in Altschul et al. (1990) J Mol Biol 215: 403-410. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1992) Proc Natl Acad Sci USA 89: 10915-10919).

In addition to calculating percent sequence identity, the BLAST algorithm also performs a statistical analysis of the similarity between two sequences (see, e.g., Karlin & Altschul (1993) Proc Nat'l Acad Sci USA 90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

### EXAMPLES

The following examples are offered to illustrate, but not to limit the claimed invention. One of skill will recognize a variety of non-critical parameters that can be changed while achieving essentially similar results.

A common problem in industrial settings is plasmid stability and retention in yeast under propagation and/or production conditions. For example, the stability of a high copy number plasmid that is currently used as a vector to overexpress genes in yeast, even in the presence of antibiotics as selective agents, was found to be less than 40%.

As described herein, the presence of an endogenous or native plasmid in a yeast strain was discovered. Sequencing of the plasmid showed more than 99% similarity to other 2 μm plasmids reported in the literature. The fact that this plasmid was identified, despite the extensive manipulations done to this strain, suggest that this native plasmid is very stable. To explore the possibility of using this plasmid as a cloning vector to overexpress genes in yeast cells, several selection agents were integrated into the plasmid by recombination. The resulting plasmid was very stable. The plasmid can be used to transform other yeast strains, such as yeast strain W303.

Previous groups have shown that the 2 μm plasmid contains only a few unique restriction endonuclease recognition sites where DNA can be cloned without affecting plasmid replication. A new region, previously ignored by other groups, into which nucleic acid sequences of interest can be introduced via homologous recombination, was discovered between the REP2 and FLP genes. Additionally, three separate sites in this region (i.e., the region between REP1 and RAF1, the region between RAF1 and STB and the region between STB and IR1) were shown to be useful sites for integration, yielding highly stable recombinant cells.

Useful applications for this technology include the use of the native 2 μm yeast plasmid of Saccharomyces as a vector to clone and/or overexpress genes of interest, e.g., genes that encode therapeutic agents or that produce pharmaceutical agents, carbon capture or degradation, saccharification, and many others, e.g., as discussed herein. The fact that 2 μm plasmids in yeast typically have about 40-100 copies per cell can increase gene expression levels of cloned genes and maintain mitotic stability of the plasmid over many generations.

Native 2 μm plasmids exist in other yeast strains and can also be similarly used as a platform for gene and library over expression. Native plasmids in yeast or filamentous fungi such as Yarrowia may also be used.

### Identification of the Presence of a Native 2 μM Endogenous Plasmid 1N Strain NRRL YB-1951

To determine whether S. cerevisiae strain NRRL YB-1952, referred to herein as RN4, contained a native 2 μm endogenous plasmid, 2 DNA segments corresponding to the coding regions of the REP1 and REP2 proteins were amplified by PCR with the following primers:

Primer REP1-F:

| | |
|---|---|
| 5′ GGTAGCTCCTGATCTCCTATATGACC 3′ | (SEQ ID NO: 2) |
| Primer REP1-R: | |
| 5′ ATGCAGCACTTCCAACCTATGGTGTACG 3′ | (SEQ ID NO: 3) |
| Primer REP2-F: | |
| 5′ GGTTCACTTCAGTCCTTCCTTCCAACTCAC 3′ | (SEQ ID NO: 4) |
| Primer REP2-R: | |
| 5′ AAAGCACGTACAGCTTATAGCGTCTGGG 3′ | (SEQ ID NO: 5) |

Using chromosomal DNA from strain RN4 as template for the PCR reactions, 2 DNA products of 567 base pairs for REP1 and 619 base pairs for REP2 were obtained. These sizes correspond exactly to the expected sizes according to the reported sequence of a 2 μm plasmid found in S. cerevisiae strain A364A (GenBank J01347.1).

### Determination of the DNA Sequence of the Native 2 μM Endogenous Plasmid Found in Strain RN4

To obtain the complete DNA sequence of the endogenous 2 μm plasmid present in RN4 strain, primers 4, 15 and 2, 10 (Table 1) were used to amplify the plasmid in two pieces using Phusion High-Fidelity polymerase (New England BioLabs) in 50 ul reactions. The resulting PCR products were separated in a 1% agarose gel (data not shown) and the DNA bands were cut and purified. The purified DNA fragments were subjected to PCR sequencing (ABI 3730×1 sequencer) using primers 1 to 20, shown in Table 1 below. The assembled sequence is shown in SEQ ID NO: 1, and a plasmid map is shown in FIG. 2. The sequence of the 2 μm plasmid from RN4 differed from the previously sequenced 2 μm strain from strain A364A (GeneBank J01347.1) at just two residues:

Nucleotide
Positions

| Strain | 385 | 707 |
|---|---|---|
| J01347 | G | T |
| RN4 | A | C |

TABLE 1
Primers used to amplify and sequence the native
2-μm endogenous plasmid present in strain RN4.

(SEQ ID NO: 6)

| | |
|---|---|
| 1 | 5′ ATGCAGCACTTCCAACCTATGGTGTACG 3′ |
| | (SEQ ID NO: 7) |
| 2 | 5′ GGTAGCTCCTGATCTCCTATATGACC 3′ |
| | (SEQ ID NO: 8) |
| 3 | 5′ AAAGCACGTACAGCTTATAGCGTCTGGG 3′ |
| | (SEQ ID NO: 9) |
| 4 | 5′ GGTTCACTTCAGTCCTTCCTTCCAACTCAC 3′ |
| | (SEQ ID NO: 10) |
| 5 | 5′ GTACACTAGTGCAGGATCAGGCCAATCC 3′ |
| | (SEQ ID NO: 11) |
| 6 | 5′ GCTCAGCAAAGGCAGTGTGATCTAAG 3′ |
| | (SEQ ID NO: 12) |
| 7 | 5′ TTTTGTTCTACAAAAATGCATCCCG 3′ |
| | (SEQ ID NO: 13) |
| 8 | 5′ AGATGCAAGTTCAAGGAGCGAAAGGTGG 3′ |
| | (SEQ ID NO: 14) |
| 9 | 5′ GGAAGGACTGAAGTGAACCATGC 3′ |
| | (SEQ ID NO: 15) |
| 10 | 5′ GTCTCTACTTCTTGTTCGCCTGGAGGG 3′ |
| | (SEQ ID NO: 16) |
| 11 | 5′ GTTGTTTTGACATGTGATCTGCACAG 3′ |
| | (SEQ ID NO: 17) |
| 12 | 5′ CGGCCGGTGCATTTTTCGAAAGAACGCG 3′ |
| | (SEQ ID NO: 18) |
| 13 | 5′ GGGCCTAACGGAGTTGACTAATGTTGTG 3′ |
| | (SEQ ID NO: 19) |
| 14 | 5′ GTTTCAGGGAAAACTCCCAGGT 3′ |
| | (SEQ ID NO: 20) |
| 15 | 5′ GGTCATATAGGAGATCAGGAGCTACC 3′ |
| | (SEQ ID NO: 21) |
| 16 | 5′ CCCAGACGCTATAAGCTGTACGTGCTTT 3′ |
| | (SEQ ID NO: 22) |
| 17 | 5′ TGTTATTCTGTAGCATCAAATCTATGG 3′ |
| | (SEQ ID NO: 23) |
| 18 | 5′ AGATTGATGTTTTTGTCCATAGTAAGG 3′ |
| | (SEQ ID NO: 24) |
| 19 | 5′ TATAAGCTGTACGTGCTTTTACCG 3′ |
| | (SEQ ID NO: 25) |
| 20 | 5′ CCACAAACTGACGAACAAGC 3′ |

SEQ ID NO: 1 provides a DNA sequence of the native 2 μm endogenous plasmid in strain RN4:

TTTGGTTTTCTTTTACCAGTATTGTTCGTTTGATAATGTATTCTTGCTT
ATTACATTATAAAATCTGTGCAGATCACATGTCAAAACAACTTTTTATC
ACAAGATAGTACCGCAAAACGAACCTGCGGGCCGTCTAAAAATTAAGGA
AAAGCAGCAAAGGTGCATTTTTAAAATATGAAATGAAGATACCGCAGTA
CCAATTATTTTCGCAGTACAAATAATGCGCGGCCGGTGCATTTTTCGAA
AGAACGCGAGACAAACAGGACAATTAAAGTTAGTTTTTCGAGTTAGCGT
GTTTGAATACTGCAAGATACAAGATAAATAGAGTAGTTGAAACTAGATA
TCAATTGCACACAAGATCGGCGCTAAGCATGCCACAATTTGATATATTA
TGTAAAACACCACCTAAGGTGCTTGTTCGTCAGTTTGTGGAAAGGTTTG
AAAGACCTTCAGGTGAGAAAATAGCATTATGTGCTGCTGAACTAACCTA
TTTATGTTGGATGATTACACATAACGGAACAGCAATCAAGAGAGCCACA
TTCATGAGCTATAATACTATCATAAGCAATTCGCTGAGTTTCGATATTG
TCAATAAATCACTCCAGTTTAAATACAAGACGCAAAAAGCAACAATTCT
GGAAGCCTCATTAAAGAAATTGATTCCTGCTTGGGAATTTACAATTATT
CCTTACTATGGACAAAAACACCAATCTGATATCACTGATATTGTAAGTA
GTTTGCAATTACAGTTCGAATCATCGGAAGAAGCAGATAAGGGAAATAG
CCACAGTAAAAAAATGCTTAAAGCACTTCTAAGTGAGGGTGAAAGCATC
TGGGAGATCACTGAGAAAATACTAAATTCGTTTGAGTATACTTCGAGAT
TTACAAAAACAAAAACTTTATACCAATTCCTCTTCCTAGCTACTTTCAT
CAATTGTGGAAGATTCAGCGATATTAAGAACGTTGATCCGAAATCATTT
AAATTAGTCCAAAATAAGTATCTGGGAGTAATAATCCAGTGTTTAGTGA
CAGAGACAAAGACAAGCGTTAGTAGGCACATATACTTCTTTAGCGCAAG
GGGTAGGATCGATCCACTTGTATATTTGGATGAATTTTTGAGGAATTCT
GAACCAGTCCTAAAACGAGTAAATAGGACCGGCAATTCTTCAAGCAATA
AACAGGAATACCAATTATTAAAAGATAACTTAGTCAGATCGTACAATAA
AGCTTTGAAGAAAAATGCGCCTTATTCAATCTTTGCTATAAAAAATGGC
CCAAAATCTCACATTGGAAGACATTTGATGACCTCATTTCTTTCAATGA
AGGGCCTAACGGAGTTGACTAATGTTGTGGGAAATTGGAGCGATAAGCG
TGCTTCTGCCGTGGCCAGGACAACGTATACTCATCAGATAACAGCAATA
CCTGATCACTACTTCGCACTAGTTTCTCGGTACTATGCATATGATCCAA
TATCAAAGGAAATGATAGCATTGAAGGATGAGACTAATCCAATTGAGGA
GTGGCAGCATATAGAACAGCTAAAGGGTAGTGCTGAAGGAAGCATACGA
TACCCCGCATGGAATGGGATAATATCACAGGAGGTACTAGACTACCTTT
CATCCTACATAAATAGACGCATATAAGTACGCATTTAAGCATAAACACG
CACTATGCCGTTCTTCTCATGTATATATATATACAGGCAACACGCAGAT
ATAGGTGCGACGTGAACAGTGAGCTGTATGTGCGCAGCTCGCGTTGCAT
TTTCGGAAGCGCTCGTTTTCGGAAACGCTTTGAAGTTCCTATTCCGAAG
TTCCTATTCTCTAGAAAGTATAGGAACTTCAGAGCGCTTTTGAAAACCA
AAAGCGCTCTGAAGACGCACTTTCAAAAAACCAAAAACGCACCGGACTG
TAACGAGCTACTAAAATATTGCGAATACCGCTTCCACAAACATTGCTCA
AAAGTATCTCTTTGCTATATATCTCTGTGCTATATCCCTATATAACCTA
CCCATCCACCTTTCGCTCCTTGAACTTGCATCTAAACTCGACCTCTACA
TCAACAGGCTTCCAATGCTCTTCAAATTTTACTGTCAAGTAGACCCATA
CGGCTGTAATATGCTGCTCTTCATAATGTAAGCTTATCTTTATCGAATC
GTGTGAAAAACTACTACCGCGATAAACCTTTACGGTTCCCTGAGATTGA
ATTAGTTCCTTTAGTATATGATACAAGACACTTTTGAACTTTGTACGAC
GAATTTTGAGGTTCGCCATCCTCTGGCTATTTCCAATTATCCTGTCGGC
TATTATCTCCGCCTCAGTTTGATCTTCCGCTTCAGACTGCCATTTTTCA
CATAATGAATCTATTTCACCCCACAATCCTTCATCCGCCTCCGCATCTT
GTTCCGTTAAACTATTGACTTCATGTTGTACATTGTTTAGTTCACGAGA
AGGGTCCTCTTCAGGCGGTAGCTCCTGATCTCCTATATGACCTTTATCC
TGTTCTCTTTCCACAAACTTAGAAATGTATTCATGAATTATGGAGCACC
TAATAACATTCTTCAAGGCGGAGAAGTTTGGGCCAGATGCCCAATATGC
TTGACATGAAAACGTGAGAATGAATTTAGTATTATTGTGATATTCTGAG
GCAATTTTATTATAATCTCGAAGATAAGAGAAGAATGCAGTGACCTTTG
TATTGACAAATGGAGATTCCATGTATCTAAAAAATACGCCTTTAGGCCT
TCTGATACCCTTTCCCCTGCGGTTTAGCGTGCCTTTTACATTAATATCT
AAACCCTCTCCGATGGTGGCCTTTAACTGACTAATAAATGCAACCGATA
TAAACTGTGATAATTCTGGGTGATTTATGATTCGATCGACAATTGTATT
GTACACTAGTGCAGGATCAGGCCAATCCAGTTCTTTTTCAATTACCGGT
GTGTCGTCTGTATTCAGTACATGTCCAACAAATGCAAATGCTAACGTTT
TGTATTTCTTATAATTGTCAGGAACTGGAAAAGTCCCCCTTGTCGTCTC
GATTACACACCTACTTTCATCGTACACCATAGGTTGGAAGTGCTGCATA
ATACATTGCTTAATACAAGCAAGCAGTCTCTCGCCATTCATATTTCAGT
TATTTTCCATTACAGCTGATGTCATTGTATATCAGCGCTGTAAAAATCT
ATCTGTTACAGAAGGTTTTCGCGGTTTTTATAAACAAAACTTTCGTTAC
GAAATCGAGCAATCACCCCAGCTGCGTATTTGGAAATTCGGGAAAAAGT
AGAGCAACGCGAGTTGCATTTTTTACACCATAATGCATGATTAACTTCG
AGAAGGGATTAAGGCTAATTTCACTAGTATGTTTCAAAAACCTCAATCT
GTCCATTGAATGCCTTATAAAACAGCTATAGATTGCATAGAAGAGTTAG
CTACTCAATGCTTTTTGTCAAAGCTTACTGATGATGATGTGTCTACTTT
CAGGCGGGTCTGTAGTAAGGAGAATGACATTATAAAGCTGGCACTTAGA
ATTCCACGGACTATAGACTATACTAGTATACTCCGTCTACTGTACGATA
CACTTCCGCTCAGGTCCTTGTCCTTTAACGAGGCCTTACCACTCTTTTG
TTACTCTATTGATCCAGCTCAGCAAAGGCAGTGTGATCTAAGATTCTAT
CTTCGCGATGTAGTAAAACTAGCTAGACCGAGAAAGAGACTAGAAATGC
AAAAGGCACTTCTACAATGGCTGCCATCATTATTATCCGATGTGACGCT
GCAGCTTCTCAATGATATTCGAATACGCTTTGAGGAGATACAGCCTAAT
ATCCGACAAACTGTTTTACAGATTTACGATCGTACTTGTTACCCATCAT
TGAATTTTGAACATCCGAACCTGGGAGTTTTCCCTGAAACAGATAGTAT
ATTTGAACCTGTATAATAATATATAGTCTAGCGCTTTACGGAAGACAAT
GTATGTATTTCGGTTCCTGGAGAAACTATTGCATCTATTGCATAGGTAA
TCTTGCACGTCGCATCCCCGGTTCATTTTCTGCGTTTCCATCTTGCACT
TCAATAGCATATCTTTGTTAACGAAGCATCTGTGCTTCATTTTGTAGAA
CAAAAATGCAACGCGAGAGCGCTAATTTTTCAAACAAAGAATCTGAGCT
GCATTTTTACAGAACAGAAATGCAACGCGAAAGCGCTATTTTACCAACG
AAGAATCTGTGCTTCATTTTTGTAAAACAAAAATGCAACGCGAGAGCGC
TAATTTTTCAAACAAAGAATCTGAGCTGCATTTTTACAGAACAGAAATG
CAACGCGAGAGCGCTATTTTACCAACAAAGAATCTATACTTCTTTTTTG
TTCTACAAAAATGCATCCCGAGAGCGCTATTTTTCTAACAAAGCATCTT
AGATTACTTTTTTTCTCCTTTGTGCGCTCTATAATGCAGTCTCTTGATA
ACTTTTTGCACTGTAGGTCCGTTAAGGTTAGAAGAAGGCTACTTTGGTG
TCTATTTTCTCTTCCATAAAAAAAGCCTGACTCCACTTCCCGCGTTTAC
TGATTACTAGCGAAGCTGCGGGTGCATTTTTTCAAGATAAAGGCATCCC
CGATTATATTCTATACCGATGTGGATTGCGCATACTTTGTGAACAGAAA
GTGATAGCGTTGATGATTCTTCATTGGTCAGAAAATTATGAACGGTTTC
TTCTATTTTGTCTCTATATACTACGTATAGGAAATGTTTACATTTTCGT
ATTGTTTTCGATTCACTCTATGAATAGTTCTTACTACAATTTTTTTGTC
TAAAGAGTAATACTAGAGATAAACATAAAAAATGTAGAGGTCGAGTTTA
GATGCAAGTTCAAGGAGCGAAAGGTGGATGGGTAGGTTATATAGGGATA
TAGCACAGAGATATATAGCAAAGAGATACTTTTGAGCAATGTTTGTGGA
AGCGGTATTCGCAATATTTTAGTAGCTCGTTACAGTCCGGTGCGTTTTT
GGTTTTTTGAAAGTGCGTCTTCAGAGCGCTTTTGGTTTTCAAAAGCGCT
CTGAAGTTCCTATACTTTCTAGAGAATAGGAACTTCGGAATAGGAACTT
CAAAGCGTTTCCGAAAACGAGCGCTTCCGAAAATGCAACGCGAGCTGCG
CACATACAGCTCACTGTTCACGTCGCACCTATATCTGCGTGTTGCCTGT
ATATATATATACATGAGAAGAACGGCATAGTGCGTGTTTATGCTTAAAT
GCGTACTTATATGCGTCTATTTATGTAGGATGAAAGGTAGTCTAGTACC
TCCTGTGATATTATCCCATTCCATGCGGGGTATCGTATGCTTCCTTCAG
CACTACCCTTTAGCTGTTCTATATGCTGCCACTCCTCAATTGGATTAGT
CTCATCCTTCAATGCTATCATTTCCTTTGATATTGGATCATACCCTAGA
AGTATTACGTGATTTTCTGCCCCTTACCCTCGTTGCTACTCTCCTTTTT
TTCGTGGGAACCGCTTTAGGGCCCTCAGTGATGGTGTTTTGTAATTTAT
ATGCTCCTCTTGCATTTGTGTCTCTACTTCTTGTTCGCCTGGAGGGAAC
TTCTTCATTTGTATTAGCATGGTTCACTTCAGTCCTTCCTTCCAACTCA
CTCTTTTTTTGCTGTAAACGATTCTCTGCCGCCAGTTCATTGAAACTAT
TGAATATATCCTTTAGAGATTCCGGGATGAATAAATCACCTATTAAAGC
AGCTTGACGATCTGGTGGAACTAAAGTAAGCAATTGGGTAACGACGCTT
ACGAGCTTCATAACATCTTCTTCCGTTGGAGCTGGTGGGACTAATAACT
GTGTACAATCCATTTTTCTCATGAGCATTTCGGTAGCTCTCTTCTTGTC
TTTCTCGGGCAATCTTCCTATTATTATAGCAATAGATTTGTATAGTTGC
TTTCTATTGTCTAACAGCTTGTTATTCTGTAGCATCAAATCTATGGCAG
CCTGACTTGCTTCTTGTGAAGAGAGCATACCATTTCCAATCGAATCAAA
CCTTTCCTTAACCATCTTCGCAGCAGGCAAAATTACCTCAGCACTGGAG
TCAGAAGATACGCTGGAATCTTCTGCGCTAGAATCAAGACCATACGGCC
TACCGGTTGTGAGAGATTCCATGGGCCTTATGACATATCCTGGAAAGAG
TAGCTCATCAGACTTACGTTTACTCTCTATATCAATATCTACATCAGGA
GCAATCATTTCAATAAACAGCCGACATACATCCCAGACGCTATAAGCTG
TACGTGCTTTTACCGTCAGATTCTTGGCTGTTTCAATGTCGTCCAT

### Integration of the KanMX Marker into the R1Site of the Native 2 μM Endogenous Plasmid of RN4

The KanMX cassette, which confers resistance to the antibiotic G418 to yeast, was integrated into the native 2 μm plasmid of strain RN4 via in vivo homologous recombination at the site 3 shown in FIG. 1. For this purpose, the KanMX cassette from an in house vector PLS1448, derived from p427TEF (DualBiosystems AG), was amplified by PCR. The primers used contained flanks of 66 bp and 68 bp homology to the integration site (underlined). The primer pair used to obtain the integration cassette was:

(SEQ ID NO: 26)
5′-ACCTGCGGGCCGTCTAAAAATTAAGGAAAAGCAGCAAAGGTGCATT
TTTAAAATATGAAATGAAGCTCACAGACGCGTTGAATTGTCCC-3′
(SEQ ID NO: 27)
5′-CGCGTTCTTTCGAAAAATGCACCGGCCGCGCATTATTTGTACTGCG
AAAATAATTGGTACTGCGGTATGGTTAAAAAATGAGCTGATTTAAC-3′

The PCR product was cleaned using a QIAGEN PCR purification kit according to manufacturer's protocol. RN4 competent cells were prepared using SIGMA YEAST-1 transformation kit protocol, and 500 ng of PCR product was used for the transformation, and selected on YPD+G418 (200 μg/mL) after 4.5 hours recovery in YPD. Two colonies from the transformation plate were used for plasmid stability studies.

### Stability Determination of the Modified 2 μM Endogenous Plasmid from RN4

The two colonies described above, were grown overnight in YPD and YPD+G418 (200 μg/mL). After 1 day, plasmid stability of the cultures were determined by plating appropriate culture dilutions onto YPD and YPD+G418 (200 μg/mL) agar plates. The plates were incubated at 30° C. for 2 days, and the colonies on the plates were counted. 2% of the overnight culture was subcultured into YPD and YPD+G418 (200 μg/mL) and was grown for 3 days. After which, plasmid stability of the cultures were determined as previously described. The native 2 μm plasmid harboring the KanMX cassette was determined to be approximately 60-80% retained. There were no differences in plasmid stability between the cultures grown in YPD versus YPD+G418 (200 μg/mL), and growth for 1 or 3 days.

### Integration of a Hygromycin Resistance Marker into R2 & R3 Sites of the Native 2 μM Endogenous Plasmid of RN4

Two new integration sites between the REP2 and FLP1 genes were selected for integration (R2 and R3 sites in FIG. 1). The hygromycin selective marker (1.8 kb) integration cassette was amplified with 65 bp flanks homologous to the 2 μm plasmid in the R2 and R3 regions (underlined) using Phusion High-fidelity polymerase in 50 ul reactions. The primer pairs used to obtain the integration cassette were:

### Region 2:

(SEQ ID NO: 28)
5′-TTATCACAAGATAGTACCGCAAAACGAACCTGCGGGCCGTCTAAAA
ATTAAGGAAAAGCAGCAAAcatctgtgcggtatttcacaccgc
(SEQ ID NO: 29)
5′-CATTATTTGTACTGCGAAAATAATTGGTACTGCGGTATCTTCATTT
CATATTTTAAAAATGCACCgaagcaaaaattacggctcct

### Region 3:

(SEQ ID NO: 30)
5′-TGTGCAGATCACATGTCAAAACAACTTTTTATCACAAGATAGTACC
GCAAAACGAACCTGCGGGCcatctgtgcggtatttcacaccgc
(SEQ ID NO: 31)
5′-ACTGCGGTATCTTCATTTCATATTTTAAAAATGCACCTTTGCTGCT
TTTCCTTAATTTTTAGACG gaagcaaaaattacggctcct

The PCR product was cleaned using a QIAGEN PCR purification kit according to manufacturer's protocol. RN4 competent cells were prepared using SIGMA YEAST-1 transformation kit protocol, and 500 ng of PCR product was used for the transformation, and selected on YPD+hygromycin (300 μg/mL) after 4 hours recovery in YPD. Three colonies from the transformation plate were used for plasmid stability studies.

Overnight cultures from colonies obtained as described above were initiated in YPD/HYG (200 ug/ml) media. The plasmid stability of the cultures were determined by plating appropriate culture dilutions onto YPD and YPD+hygromycin (300 μg/mL) agar plates. Afterward the culture was diluted 1 in 100 in YPD with no antibiotics and incubated at 30° C., at 24 and 48 hrs samples for retention studies were taken and retention was tested as above. The retention of the plasmids carrying the hygromycin resistance marker in both regions in RN4 strain was about 90% after 24 hrs and more than 80% after 48 hrs with no selection pressure (FIG. 3).

### Integration of the Larger Fragment (4 Kb) with Two Orfs into R2 & R3Sites of the Native 2 μM Endogenous Plasmid of RN4

To check retention of a larger insert, a Gene1/Gateway/SAT 1 marker cassette (4 kb size) was amplified for integration into R2 and R3 of the endogenous 2 μm plasmid of RN4 (R2 & R3 sites in FIG. 1). The 4 kb integration cassette was amplified with 65 bp flanks homologous to the 2 μm plasmid in R2 and R3 regions (underlined) using Phusion High-fidelity polymerase in 50 ul reactions. Primers used to obtain the integration cassette were:

### Region 2:

(SEQ ID NO: 32)
5′-TTATCACAAGATAGTACCGCAAAACGAACCTGCGGGCCGTCTAAAA
ATTAAGGAAAAGCAGCAAA gggaacaaaagctggagctccatagc
(SEQ ID NO: 33)
5′-CATTATTTGTACTGCGAAAATAATTGGTACTGCGGTATCTTCATTT
CATATTTTAAAAATGCACCgaagcaaaaattacggctcct

### Region 3:

(SEQ ID NO: 34)
5′-TGTGCAGATCACATGTCAAAACAACTTTTTATCACAAGATAGTACC
GCAAAACGAACCTGCGGGC gggaacaaaagctggagctccatagc
(SEQ ID NO: 35)
5′-ACTGCGGTATCTTCATTTCATATTTTAAAAATGCACCTTTGCTGCT
TTTCCTTAATTTTTAGACG gaagcaaaaattacggctcct

The PCR product was cleaned using a QIAGEN PCR purification kit according to manufacturer's protocol. RN4 competent cells were prepared using SIGMA YEAST-1 transformation kit protocol, and 500 ng of PCR product was used for the transformation, and selected on YPD+ClonNAT (100 μg/mL) after 4 hours recovery in YPD (ClonNat is the common trade name for the natural product nourseothricin; the relevant marker gene is streptothricin acetyltransferase 1 (sat 1)). Three colonies from the transformation plate were used for plasmid stability studies.

### Stability of the Large Insert in Sites R2 and R3

Colonies were grown overnight in YPD+ClonNAT 200 ug/ml, and after 24 hrs samples were taken for retention study and to start new cultures in YPD with no selection. The plasmid stability of the cultures was determined by plating appropriate culture dilutions onto YPD and YPD+ClonNAT 200 ug/ml and the same cultures were rediluted 1/100 in fresh YPD with no antibiotics to initiate new cultures. The same procedure used for generation of additional generations without selection. The retention in both regions in RN4 strain was about 90% after 24 hrs and first subculture and more than 80% after second serial subculture with no selection pressure (FIGS. 3 and 4).

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method of making a recombinant plasmid in a yeast cell, the method comprising:
providing the yeast cell, which yeast cell comprises a stable 2 μm plasmid;
introducing a heterologous nucleic acid into the yeast cell, which heterologous nucleic acid comprises recombination sites flanking a subsequence encoding a selectable marker; and,
permitting integration of the selectable marker into the 2 μm plasmid via homologous recombination between the recombination sites and the plasmid, wherein the homologous recombination occurs between subsequences of the 2 μm plasmid that encode FLP and REP2, thereby producing a recombinant plasmid in the yeast cell.

2. The method of claim 1, wherein the 2 μm plasmid is a wild-type 2 μm plasmid endogenous to the yeast cell.

3. The method of claim 1, wherein the yeast cell is a Saccharomyces cell.

4. The method of claim 1, wherein the method comprises:
(a) introducing the 2 μm plasmid into the yeast cell;
(b) assembling the heterologous nucleic acid via PCR, by direct synthesis, or both; or
(c) introducing a pooled population of variant heterologous nucleic acids into a population of yeast cells, and selecting the population of yeast cells for one or more activity of interest.

5. The method of claim 4(c), wherein the pooled population of variant heterologous nucleic acids are produced by splicing by overlap extension (SOE) PCR, direct synthesis, or a combination thereof.

6. The method of claim 1, comprising culturing the yeast cell under selective conditions after said permitting, thereby selecting progeny of the yeast cell based upon expression of the selectable marker.

7. The method of claim 6, wherein the selective conditions:
(a) are continuously maintained during growth phase;
(b) comprise non-permissive auxotrophic growth conditions, said selectable marker comprising an auxotrophic growth agent; or
(c) comprise culturing the yeast cell in the presence of an antibiotic, an antifungal, or a toxin, the selectable marker comprising a resistance agent to the antibiotic, the antifungal, or the toxin.

8. The method of claim 6, wherein the selectable marker provides hygromycin resistance to the yeast cell.

9. The method of claim 6, comprising isolating copies of the recombinant plasmid from the progeny and introducing one or more of the copies into one or more additional cell(s).

10. The method of claim 6, wherein culturing the yeast cell under selective conditions results in progeny yeast cells comprising at least 5 copies of the recombinant plasmid per cell.

11. The method of claim 1, wherein the heterologous nucleic acid further comprises a gene or expression cassette that encodes a polypeptide or RNA product of interest.

12. The method of claim 11, wherein the polypeptide of interest comprises an enzyme.

13. The method of claim 12, wherein the enzyme comprises a dehydrogenase, a dehydratase, or an invertase.

14. The method of claim 12, wherein the enzyme catalyzes or regulates degradation or synthesis of a sugar, a polysaccharide, a cellulosic material, a polymer, a chemical compound, a fatty acid, a fatty alcohol, a ketone, a lipid, an organic acid, or succinate, or wherein the polypeptide of interest regulates expression, synthesis, or folding of an additional polypeptide that catalyzes or regulates degradation or synthesis a sugar, a polysaccharide, a cellulosic material, a polymer, a chemical compound, a fatty acid, a fatty alcohol, a ketone, a lipid, an organic acid, or succinate.

15. A method of producing a protein, the method comprising culturing the yeast cell of claim 1.

16. A composition comprising a stable recombinant yeast 2 μm plasmid comprising a heterologous nucleic acid subsequence between an FLP and a REP2 gene of the plasmid.

17. The composition of claim 16, wherein the plasmid:
(a) comprises a subsequence that is at least 90% identical to a full-length endogenous 2 μm plasmid sequence (SEQ ID NO:1);
(b) is free of a bacterial origin of replication;
(c) encodes functional REP1, REP2 and FLP proteins; or
(d) comprises a complete set of native 2 μm plasmid coding and regulatory sequences; or
(e) is stably propagated in a yeast cell culture comprising a selection agent that selects for an expression product of the heterologous nucleic acid subsequence.

18. The composition of claim 17(e), comprising the yeast cell culture and the selection agent, the expression product comprising selection agent resistance activity, wherein the selection agent is present in the composition at a concentration sufficient to exert selective pressure on cells of the culture to stably retain the plasmid.

19. The composition of claim 18, wherein the selection agent is an antifungal agent, an antibiotic agent, or a toxin.

20. The composition of claim 16, wherein the heterologous nucleic acid encodes a selectable marker.

21. The composition of claim 20, wherein the heterologous nucleic acid additionally encodes a polypeptide or RNA product of interest.

22. The composition of claim 21, wherein the polypeptide is an enzyme.

23. The composition of claim 22, wherein the enzyme catalyzes or regulates degradation or synthesis of a sugar, a polysaccharide, a cellulosic material, a polymer, a chemical compound, a fatty acid, a fatty alcohol, a ketone, a lipid, an organic acid, or succinate, or wherein the polypeptide or target RNA product regulates expression, synthesis, or folding of an additional polypeptide that catalyzes or regulates degradation or synthesis a sugar, a polysaccharide, a cellulosic material, a polymer, a chemical compound, a fatty acid, a fatty alcohol, a ketone, a lipid, an organic acid, or succinate.

24. The composition of claim 16, comprising a yeast cell culture, wherein the yeast cell culture is an auxotrophic cell culture and the plasmid encodes an auxotrophic agent that increases a rate of growth of cells in the culture under non-permissive auxotrophic growth conditions.

25. The composition of claim 16, comprising a yeast cell comprising the plasmid.

26. The composition of claim 25, wherein the yeast cell
(a) comprises at least 5 copies of the plasmid; or
(b) is a Saccharomyces cell.


SEQUENCE LISTING (35 sequences)

SEQ ID NO: 1 - DNA, 6318 bp, Saccharomyces cerevisiae
tttggttttcttttaccagtattgttcgtttgataatgtattcttgcttattacattataaaatctgtgcagatcacatgtcaaaacaactttttatcacaagatagtaccgcaaaacgaacctgcgggccgtctaaaaattaaggaaaagcagcaaaggtgcatttttaaaatatgaaatgaagataccgcagtaccaattattttcgcagtacaaataatgcgcggccggtgcatttttcgaaagaacgcgagacaaacaggacaattaaagttagtttttcgagttagcgtgtttgaatactgcaagatacaagataaatagagtagttgaaactagatatcaattgcacacaagatcggcgctaagcatgccacaatttgatatattatgtaaaacaccacctaaggtgcttgttcgtcagtttgtggaaaggtttgaaagaccttcaggtgagaaaatagcattatgtgctgctgaactaacctatttatgttggatgattacacataacggaacagcaatcaagagagccacattcatgagctataatactatcataagcaattcgctgagtttcgatattgtcaataaatcactccagtttaaatacaagacgcaaaaagcaacaattctggaagcctcattaaagaaattgattcctgcttgggaatttacaattattccttactatggacaaaaacaccaatctgatatcactgatattgtaagtagtttgcaattacagttcgaatcatcggaagaagcagataagggaaatagccacagtaaaaaaatgcttaaagcacttctaagtgagggtgaaagcatctgggagatcactgagaaaatactaaattcgtttgagtatacttcgagatttacaaaaacaaaaactttataccaattcctcttcctagctactttcatcaattgtggaagattcagcgatattaagaacgttgatccgaaatcatttaaattagtccaaaataagtatctgggagtaataatccagtgtttagtgacagagacaaagacaagcgttagtaggcacatatacttctttagcgcaaggggtaggatcgatccacttgtatatttggatgaatttttgaggaattctgaaccagtcctaaaacgagtaaataggaccggcaattcttcaagcaataaacaggaataccaattattaaaagataacttagtcagatcgtacaataaagctttgaagaaaaatgcgccttattcaatctttgctataaaaaatggcccaaaatctcacattggaagacatttgatgacctcatttctttcaatgaagggcctaacggagttgactaatgttgtgggaaattggagcgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacatcaacaggcttccaatgctcttcaaattttactgtcaagtagacccatacggctgtaatatgctgctcttcataatgtaagcttatctttatcgaatcgtgtgaaaaactactaccgcgataaacctttacggttccctgagattgaattagttcctttagtatatgatacaagacacttttgaactttgtacgacgaattttgaggttcgccatcctctggctatttccaattatcctgtcggctattatctccgcctcagtttgatcttccgcttcagactgccatttttcacataatgaatctatttcaccccacaatccttcatccgcctccgcatcttgttccgttaaactattgacttcatgttgtacattgtttagttcacgagaagggtcctcttcaggcggtagctcctgatctcctatatgacctttatcctgttctctttccacaaacttagaaatgtattcatgaattatggagcacctaataacattcttcaaggcggagaagtttgggccagatgcccaatatgcttgacatgaaaacgtgagaatgaatttagtattattgtgatattctgaggcaattttattataatctcgaagataagagaagaatgcagtgacctttgtattgacaaatggagattccatgtatctaaaaaatacgcctttaggccttctgataccctttcccctgcggtttagcgtgccttttacattaatatctaaaccctctccgatggtggcctttaactgactaataaatgcaaccgatataaactgtgataattctgggtgatttatgattcgatcgacaattgtattgtacactagtgcaggatcaggccaatccagttctttttcaattaccggtgtgtcgtctgtattcagtacatgtccaacaaatgcaaatgctaacgttttgtatttcttataattgtcaggaactggaaaagtcccccttgtcgtctcgattacacacctactttcatcgtacaccataggttggaagtgctgcataatacattgcttaatacaagcaagcagtctctcgccattcatatttcagttattttccattacagctgatgtcattgtatatcagcgctgtaaaaatctatctgttacagaaggttttcgcggtttttataaacaaaactttcgttacgaaatcgagcaatcaccccagctgcgtatttggaaattcgggaaaaagtagagcaacgcgagttgcattttttacaccataatgcatgattaacttcgagaagggattaaggctaatttcactagtatgtttcaaaaacctcaatctgtccattgaatgccttataaaacagctatagattgcatagaagagttagctactcaatgctttttgtcaaagcttactgatgatgatgtgtctactttcaggcgggtctgtagtaaggagaatgacattataaagctggcacttagaattccacggactatagactatactagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcataccctagaagtattacgtgattttctgccccttaccctcgttgctactctcctttttttcgtgggaaccgctttagggccctcagtgatggtgttttgtaatttatatgctcctcttgcatttgtgtctctacttcttgttcgcctggagggaacttcttcatttgtattagcatggttcacttcagtccttccttccaactcactctttttttgctgtaaacgattctctgccgccagttcattgaaactattgaatatatcctttagagattccgggatgaataaatcacctattaaagcagcttgacgatctggtggaactaaagtaagcaattgggtaacgacgcttacgagcttcataacatcttcttccgttggagctggtgggactaataactgtgtacaatccatttttctcatgagcatttcggtagctctcttcttgtctttctcgggcaatcttcctattattatagcaatagatttgtatagttgctttctattgtctaacagcttgttattctgtagcatcaaatctatggcagcctgacttgcttcttgtgaagagagcataccatttccaatcgaatcaaacctttccttaaccatcttcgcagcaggcaaaattacctcagcactggagtcagaagatacgctggaatcttctgcgctagaatcaagaccatacggcctaccggttgtgagagattccatgggccttatgacatatcctggaaagagtagctcatcagacttacgtttactctctatatcaatatctacatcaggagcaatcatttcaataaacagccgacatacatcccagacgctataagctgtacgtgcttttaccgtcagattcttggctgtttcaatgtcgtccat

SEQ ID NO: 2 - DNA, 26 bp, Artificial Sequence
  Synthesized oligonucleotide for PCR
ggtagctcctgatctcctatatgacc

SEQ ID NO: 3 - DNA, 28 bp, Artificial Sequence
  Synthesized oligonucleotide for PCR
atgcagcacttccaacctatggtgtacg

SEQ ID NO: 4 - DNA, 30 bp, Artificial Sequence
  Synthesized oligonucleotide for PCR
ggttcacttcagtccttccttccaactcac

SEQ ID NO: 5 - DNA, 28 bp, Artificial Sequence
  Synthesized oligonucletoide for PCT
aaagcacgtacagcttatagcgtctggg

SEQ ID NO: 6 - DNA, 28 bp, Artificial Sequence
  Synthesized oligonucleotide for PCR
atgcagcacttccaacctatggtgtacg

SEQ ID NO: 7 - DNA, 26 bp, Artificial Sequence
  Synthesized oligonucleotide for PCR
ggtagctcctgatctcctatatgacc

SEQ ID NO: 8 - DNA, 28 bp, Artificial Sequence
  Synthesized oligonucleotide for PCR
aaagcacgtacagcttatagcgtctggg

SEQ ID NO: 9 - DNA, 30 bp, Artificial Sequence; identical to SEQ ID NO: 4
  Synthesized oligonucleotide for PCR

SEQ ID NO: 10 - DNA, 28 bp, Artificial Sequence
  Synthesized oligonucleotide for PCR
gtacactagtgcaggatcaggccaatcc

SEQ ID NO: 11 - DNA, 26 bp, Artificial Sequence
  Synthesized oligonucleotide for PCR
gctcagcaaaggcagtgtgatctaag

SEQ ID NO: 12 - DNA, 25 bp, Artificial Sequence
  Synthesized oligonucleotide for PCR
ttttgttctacaaaaatgcatcccg

SEQ ID NO: 13 - DNA, 28 bp, Artificial Sequence
  Synthesized oligonucleotide for PCR
agatgcaagttcaaggagcgaaaggtgg

SEQ ID NO: 14 - DNA, 23 bp, Artificial Sequence
  Synthesized oligonucleotide for PCR
ggaaggactgaagtgaaccatgc

SEQ ID NO: 15 - DNA, 27 bp, Artificial Sequence
  Synthesized oligonucleotide for PCR
gtctctacttcttgttcgcctggaggg

SEQ ID NO: 16 - DNA, 26 bp, Artificial Sequence
  Synthesized oligonucleotide for PCR
gttgttttgacatgtgatctgcacag

SEQ ID NO: 17 - DNA, 28 bp, Artificial Sequence
  Synthesized oligonucleotide for PCR
cggccggtgcatttttcgaaagaacgcg

SEQ ID NO: 18 - DNA, 28 bp, Artificial Sequence
  Synthesized oligonucleotide for PCR
gggcctaacggagttgactaatgttgtg

SEQ ID NO: 19 - DNA, 22 bp, Artificial Sequence
  Synthesized oligonucleotide for PCR
gtttcagggaaaactcccaggt

SEQ ID NO: 20 - DNA, 26 bp, Artificial Sequence
  Synthesized oligonucleotide for PCR
ggtcatataggagatcaggagctacc

SEQ ID NO: 21 - DNA, 28 bp, Artificial Sequence
  Synthesized oligonucleotide for PCR
cccagacgctataagctgtacgtgcttt

SEQ ID NO: 22 - DNA, 27 bp, Artificial Sequence
  Synthesized oligonucleotide for PCR
tgttattctgtagcatcaaatctatgg

SEQ ID NO: 23 - DNA, 27 bp, Artificial Sequence
  Synthesized oligonucleotide for PCR
agattgatgtttttgtccatagtaagg

SEQ ID NO: 24 - DNA, 24 bp, Artificial Sequence
  Synthesized oligonucleotide for PCR
tataagctgtacgtgcttttaccg

SEQ ID NO: 25 - DNA, 20 bp, Artificial Sequence
  Synthesized oligonucleotide for PCR
ccacaaactgacgaacaagc

SEQ ID NO: 26 - DNA, 89 bp, Artificial Sequence
  Synthesized oligonucleotide for PCR
acctgcgggccgtctaaaaattaaggaaaagcagcaaaggtgcatttttaaaatatgaaatgaagctcacagacgcgttgaattgtccc

SEQ ID NO: 27 - DNA, 92 bp, Artificial Sequence
  Synthesized oligonucleotide for PCR
cgcgttctttcgaaaaatgcaccggccgcgcattatttgtactgcgaaaataattggtactgcggtatggttaaaaaatgagctgatttaac

SEQ ID NO: 28 - DNA, 89 bp, Artificial Sequence
  Synthesized oligonucleotide for PCR
ttatcacaagatagtaccgcaaaacgaacctgcgggccgtctaaaaattaaggaaaagcagcaaacatctgtgcggtatttcacaccgc

SEQ ID NO: 29 - DNA, 86 bp, Artificial Sequence
  Synthesized oligonucleotide for PCR
cattatttgtactgcgaaaataattggtactgcggtatcttcatttcatattttaaaaatgcaccgaagcaaaaattacggctcct

SEQ ID NO: 30 - DNA, 89 bp, Artificial Sequence
  Synthesized oligonucleotide for PCR
tgtgcagatcacatgtcaaaacaactttttatcacaagatagtaccgcaaaacgaacctgcgggccatctgtgcggtatttcacaccgc

SEQ ID NO: 31 - DNA, 86 bp, Artificial Sequence
  Synthesized oligonucleotide for PCR
actgcggtatcttcatttcatattttaaaaatgcacctttgctgcttttccttaatttttagacggaagcaaaaattacggctcct

SEQ ID NO: 32 - DNA, 91 bp, Artificial Sequence
  Synthesized oligonucleotide for PCR
ttatcacaagatagtaccgcaaaacgaacctgcgggccgtctaaaaattaaggaaaagcagcaaagggaacaaaagctggagctccatagc

SEQ ID NO: 33 - DNA, 86 bp, Artificial Sequence; identical to SEQ ID NO: 29
  Synthesized oligonucleotide for PCR

SEQ ID NO: 34 - DNA, 91 bp, Artificial Sequence
  Synthesized oligonucleotide for PCR
tgtgcagatcacatgtcaaaacaactttttatcacaagatagtaccgcaaaacgaacctgcgggcgggaacaaaagctggagctccatagc

SEQ ID NO: 35 - DNA, 86 bp, Artificial Sequence; identical to SEQ ID NO: 31
  Synthesized oligonucleotide for PCR